(19) United States
(12) Patent Application Publication
(10) Pub. No.: US 2025/0153133 A1
(43) Pub. Date: May 15, 2025

(54) PRODUCTION SYSTEM AND PRODUCTION METHOD FOR FLAVAN OLIGOMER

(71) Applicant: HITACHI PLANT SERVICES CO., LTD., Tokyo (JP)

(72) Inventors: Yukako ASANO, Tokyo (JP); Masashi TAGAMI, Tokyo (JP); Ken OHMORI, Tokyo (JP); Naoki NARITA, Tokyo (JP)

(73) Assignee: HITACHI PLANT SERVICES CO., LTD., Tokyo (JP)

(21) Appl. No.: 18/841,056

(22) PCT Filed: Feb. 13, 2023
(86) PCT No.: PCT/JP2023/004768

§ 371 Date: Aug. 23, 2024

(30) Foreign Application Priority Data
Mar. 8, 2022 (JP) 2022-035671

(51) Int. Cl. B01J 19/00; C08G 61/12

(52) U.S. Cl. CPC B01J 19/0093; C08G 61/12

(57) ABSTRACT

The present invention provides a flavan oligomer production system and a flavan oligomer production method that can efficiently synthesize a flavan oligomer containing flavan derivatives bonded to each other at a desired degree of polymerization in high yield. A flavan oligomer production system includes a microreactor which mixes in a flow path a first fluid introduced from one of inlets and a second fluid introduced from another of the inlets, a first container in which the first fluid is disposed, a second container in which the second fluid is disposed, and a recovery container which recovers a product fluid. The first fluid is a liquid containing a flavan derivative including a flavan skeleton. The second fluid is a liquid containing a Lewis acid. The product fluid is recovered in a liquid containing a base. A flavan oligomer production method mixes a first fluid and a second fluid in a microreactor, activates a flavan derivative of the first fluid by using a Lewis acid of the second fluid, initiates a reaction between an activated flavan derivative and a flavan derivative acting as a nucleophile, and stops a reaction by using a base.

### TECHNICAL FIELD

The present invention relates to a flavan oligomer production system for producing a flavan oligomer in which flavan derivatives having a flavan skeleton are condensed with each other, and to a flavan oligomer production method.

### BACKGROUND ART

Plants contain many flavonoids as secondary metabolites. Flavonoids are a group of compounds having a flavan skeleton and belong to polyphenols. As flavonoids, various analogs have been found that differ in the binding position and number of substituents to the flavan skeleton, modification structures such as sugar chains, and three-dimensional structures. In addition, oligomers in which monomers having a flavan skeleton are condensed with each other have been found in plants.

Flavonoids are classified into flavanols, flavanones, flavones, isoflavones, anthocyanins, and the like. Flavanols include catechin, epicatechin, gallocatechin, epigallocatechin, and the like. Flavanones include naringenin, hesperetin, eriodictyol, and the like. Flavones include apigenin, luteolin, and the like. Isoflavones include genistein, daidzein, and the like. Anthocyanins include pelargonidin, cyanidin, delphinidin, and the like.

Flavonoids are known to exhibit various physiological activities such as antibacterial activity, growth inhibitory activity, antioxidant action, anticancer action, and metabolism promoting action. In addition to the activity and kinetics of oligomers, research is also underway on flavan derivatives based on the structure of natural flavonoids. Flavan derivatives and their oligomers have great potential as an undeveloped chemical space and are expected to be lead compounds for new pharmaceuticals and the like.

Flavanols have a flavan-3-ol skeleton. Flavan oligomers in which flavan-3-ols such as catechin and epicatechin are condensed are known as procyanidins. Flavan oligomers are polymers having various degrees of polymerization in plants and exist as mixtures of dimers, trimers, oligomers, and the like.

At present, in research and utilization of flavan oligomers, when attempting to separate and purify flavan oligomers from natural products with high purity, a great deal of cost and labor is required. In addition, commercially available flavan oligomers are limited in variety and are expensive. Under such circumstances, an efficient synthesis method for flavan oligomers bonded with a predetermined degree of polymerization has been desired.

Patent Literature 1 describes a method for synthesizing flavan oligomers using a flavan-3-oxo derivative and a flavan-3-ol derivative as raw materials (see paragraphs 0074 to 0077). A flavan-3-oxo derivative (0.145 mmol) and a flavan-3-ol derivative (0.435 mmol) are used as raw materials, and in tetrahydrofuran (THF), silver tetrafluoroborate (AgBF₄) (1.1 mmol) is used as a reaction catalyst and refluxed at room temperature for 4 hours. As a result, the target dimer, which is a proanthocyanidin adduct, is produced with a yield of 70%.

Patent Literature 2 describes a method for synthesizing flavan oligomers using epicatechin as a raw material (see paragraph 0052). An epicatechin derivative (monomer) in which the hydroxy group is protected is used as a raw material, and the reaction is carried out in methylene chloride at room temperature (20° C.) for 1.5 hours using zinc triflate (Zn(OTf)₂) (0.7 equivalent) as a reaction catalyst. As a result, a dimer condensate (dimer) is produced with a yield of about 58%.

Patent Literature 3 describes a method for synthesizing flavan oligomers using epigallocatechin as a raw material (see paragraph 0103). An epigallocatechin derivative (monomer) in which the hydroxy group is protected is used as a raw material, and the reaction is carried out at room temperature (20° C.) for 2 hours using zinc triflate (Zn(OTf)₂) (0.8 equivalent) as a reaction catalyst. Then, the produced dimer condensate (dimer) is isolated and purified, and the reaction is carried out at room temperature (20° C.) for 20 hours using ytterbium triflate (Yb(OTf)₃) (5 equivalents) as a reaction catalyst. As a result, a tetramer condensate (tetramer) is produced with a yield of 45%.

On the other hand, in recent years, the use of microreactors has been progressing in the fields of biotechnology, pharmaceuticals, chemicals, and the like. A microreactor is a flow-type reactor having microchannels on the order of m and is used for mixing and reacting fluids. Microreactors are fabricated using microfabrication techniques such as molding and lithography.

A feature of the synthesis reaction using a microreactor is that molecular diffusion under laminar flow becomes dominant. As the size of the reaction field decreases, molecular diffusion under laminar flow is promoted, so that fluids can be mixed uniformly and rapidly. Since the surface area relative to the volume of the fluid becomes relatively large, the surface effect and heat transfer coefficient increase, enabling rapid mixing, control of the reaction ratio, precise temperature control, and the like. Compared to conventional synthesis reactions using batch methods, it is possible to shorten the reaction time and improve the yield, so that improvement in production efficiency is expected.

### CITATION LIST

### Patent Literature

Patent Literature 1: JP5550639B
Patent Literature 2: JP2017-001982A
Patent Literature 3: JP2019-151583A

### SUMMARY OF INVENTION

### Technical Problem

Conventionally, flavan oligomers in which flavan derivatives having a flavan skeleton are condensed with each other are often synthesized by a batch method. However, when using the batch method, it is difficult to precisely control the reaction ratio of the reactants and the reaction time. Conventional synthesis methods have a problem in that the polymerization reaction continues, so that a flavan oligomer in which flavan derivatives are bonded with a desired degree of polymerization cannot be synthesized in high yield.

In conventional synthesis methods, reactants react at unintended ratios, or reactants activated by a catalyst react with products, resulting in the production of mixtures of various degrees of polymerization. In such cases, there is a problem that the separation and purification after the reaction requires cost and labor. In addition, there is a method of using an excess amount of catalyst to increase the yield, but using an excess amount of catalyst leads to catalyst loss and increased purification costs.

In the synthesis method described in Patent Literature 1, the flavan-3-ol derivative is reacted with the flavan-3-oxo derivative in a large excess of 3 equivalents. Further, silver tetrafluoroborate (AgBF₄) is added in a large excess of 7.5 equivalents. In such a synthesis method, the separation and purification after the reaction requires cost and labor, and the production efficiency of the oligomer having the desired degree of polymerization is deteriorated.

In the synthesis methods described in Patent Literatures 2 and 3, the amount of zinc triflate (Zn(OTf)₂) is small, but even at room temperature, a long reaction time of 1.5 hours or 2 hours is required. Further, zinc triflate (Zn(OTf)₂) and ytterbium triflate (Yb(OTf)₃) may produce solids, which may block the flow path when a microreactor having a microchannel is used.

Therefore, an object of the present invention is to provide a flavan oligomer production system and a flavan oligomer production method that can efficiently synthesize a flavan oligomer in which flavan derivatives are bonded at a desired degree of polymerization in high yield.

### Solution to Problem

In order to achieve the above object, the flavan oligomer production system according to the present invention is a system for producing a flavan oligomer containing flavan derivatives which include a flavan skeleton and which are bonded to each other. The flavan oligomer production system includes at least one microreactor which includes two inlets allowing fluids to be introduced into the two inlets and a flow path allowing the fluids to merge in the flow path and which mixes in the flow path a first fluid to be introduced from one of the inlets and a second fluid to be introduced from another of the inlets. The flavan oligomer production system further includes a first container in which the first fluid is to be disposed, a second container in which the second fluid is to be disposed, and a recovery container which recovers a product fluid to be produced in the at least one microreactor. The first fluid is a liquid containing a flavan derivative including a flavan skeleton. The second fluid is a liquid containing a Lewis acid. The product fluid includes an oligomer containing the flavan derivatives bonded to each other and is to be recovered in a liquid in the recovery container, the liquid containing a base.

Further, the flavan oligomer production method according to the present invention is a method for producing a flavan oligomer containing flavan derivatives which include a flavan skeleton and which are bonded to each other. The flavan oligomer production method includes, in a microreactor system including at least one microreactor which includes two inlets allowing fluids to be introduced into the two inlets and a flow path allowing the fluids to merge in the flow path and which mixes in the flow path a first fluid to be introduced from one of the inlets and a second fluid to be introduced from another of the inlets, a first container in which the first fluid is to be disposed, a second container in which the second fluid is to be disposed, and a recovery container which recovers a product fluid to be produced in the at least one microreactor, preparing as the first fluid a liquid containing a flavan derivative including a flavan skeleton, preparing as the second fluid a liquid containing a Lewis acid, mixing the first fluid and the second fluid in the microreactor, activating a part of the flavan derivative of the first fluid by using the Lewis acid of the second fluid, initiating a reaction between an activated part of the flavan derivative of the first fluid and a remaining part of the flavan derivative of the first fluid acting as a nucleophile, recovering in a liquid containing a base the product fluid being reacting, stopping a reaction of the product fluid by using the base, producing an oligomer containing the flavan derivatives bonded to each other.

### Advantageous Effects of Invention

According to the present invention, it is possible to efficiently synthesize a flavan oligomer in which flavan derivatives are bonded at a desired degree of polymerization in high yield.

### BRIEF DESCRIPTION OF DRAWINGS

FIG. 1 is a schematic diagram of a flavan oligomer production system according to the first embodiment.

FIG. 2 is a diagram showing an example of a microreactor.

FIG. 3 is a schematic diagram of a flavan oligomer production system according to the second embodiment.

FIG. 4 is a schematic diagram of a flavan oligomer production system according to the third embodiment.

FIG. 5 is a schematic diagram of a flavan oligomer production system according to the fourth embodiment.

FIG. 6 is a schematic diagram of a flavan oligomer production system according to the fifth embodiment.

### DESCRIPTION OF EMBODIMENTS

Hereinafter, a flavan oligomer production system and a flavan oligomer production method according to an embodiment of the present invention will be described with reference to the drawings. In the following drawings, the same components will be denoted by the same reference numerals, and redundant description will be omitted.

The flavan oligomer production system according to this embodiment is a system for synthesizing a flavan oligomer in which flavan derivatives having a flavan skeleton are bonded to each other. In this production system, a flavan derivative having a predetermined molecular structure is used as a starting material for the synthesis reaction. Further, a Lewis acid is used as a reaction catalyst.

The flavan derivative of the starting material is activated by a Lewis acid, becomes cationized, and becomes an electrophile. The flavan derivatives condense with each other by the reaction of the activated flavan derivative and the unactivated flavan derivative acting as a nucleophile. By such a polymerization reaction, a flavan oligomer in which flavan derivatives are bonded to each other can be produced.

In the flavan oligomer production system, a microreactor is used for the reaction of a flavan derivative and a Lewis acid, the reaction of an activated flavan derivative and an unactivated flavan derivative acting as a nucleophile, and the like. In the microreactor, fluids containing reactants are introduced into microchannels that are micro reaction fields, and these are mixed in the microchannels to initiate the reaction. Thereafter, the Lewis acid is neutralized with a base to stop the reaction.

By using a microreactor, the reaction amount of reactants such as flavan derivatives and Lewis acids, the initiation time of the reaction, and the end time of the reaction can be precisely controlled. Precise control of the reaction ratio of the reactants and the reaction time is possible. Therefore, a flavan oligomer in which flavan derivatives are bonded with a desired degree of polymerization can be synthesized in high yield.

As the starting flavan derivative, a monomer of a flavan derivative having one flavan skeleton or an oligomer of a flavan derivative having two or more flavan skeletons can be used. The flavan skeleton is represented by the following formula (a).

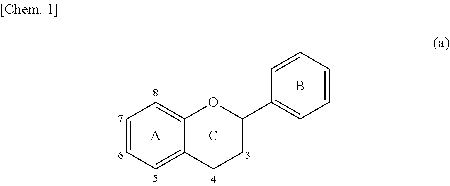

In the present specification, the oligomer of the flavan derivative means an i-mer (i is an integer of 2 or larger than 2) in which multiple monomers of the flavan derivative are condensed, such as a dimer in which two monomers of the flavan derivative are condensed and a trimer in which three monomers of the flavan derivative are condensed. Oligomers of flavan derivatives also include so-called polymers in which a large number of monomers are condensed.

The monomer of the flavan derivative and the oligomer of the flavan derivative may be a natural compound isolated from a natural product, or may be a synthetic compound chemically synthesized. The synthetic compound, which is an oligomer of the flavan derivative, can be synthesized, for example, by using the flavan oligomer production system according to this embodiment with a monomer of the flavan derivative or the like as a starting material.

As the starting flavan derivative, a derivative in which a protecting group is introduced to a substituent such as a hydroxy group may be used in order to prevent an unintended reaction. Further, depending on the reactivity and the like, a derivative to which a protecting group is not introduced may be used. The raw material liquid containing the flavan derivative can be prepared by dissolving a monomer of the flavan derivative at a predetermined concentration or an oligomer of the flavan derivative at a predetermined concentration in an appropriate solvent serving as a reaction solvent.

The protection of the hydroxy group can be carried out, for example, using a base or a protecting group-introducing compound under a polar solvent. Examples of the base include sodium hydride, alkylamide, pyridine, and the like. Examples of the protecting group-introducing compound include halogenated hydrocarbons, acyl halide compounds, silyl halide compounds, and the like.

The starting flavan derivative is preferably a flavan-3-ol derivative having a substituent derived from a hydroxy group at the 3-position carbon of the C ring. Further, a derivative having a substituent derived from a hydroxy group at the 5-position and 7-position carbons of the A ring is more preferable. For example, flavanols such as (+)-catechin, (−)-catechin, (+)-epicatechin, (−)-epicatechin, and derivatives thereof can be preferably used as starting materials.

Further, the starting flavan derivative is more preferably a derivative having a leaving group at the 4-position carbon of the C ring. Further, a derivative having a substituent having an electron donating moiety at the 3-position carbon of the C ring is more preferable. With such a structure, the 4-position carbon of the flavan skeleton can be regioselectively activated by a Lewis acid.

Further, as the starting flavan derivative, a derivative in which an electron donating group is bonded to the 5-position or 7-position carbon of the A ring is more preferable. Further, a derivative in which an electron donating group is not bonded to the 6-position carbon of the A ring is more preferable. With such a structure, the 4-position carbon of the flavan skeleton can be activated to condense the 4-position and 8′-position of the flavan skeleton.

The monomer of the starting flavan derivative is more preferably a compound represented by the following general formula (1).

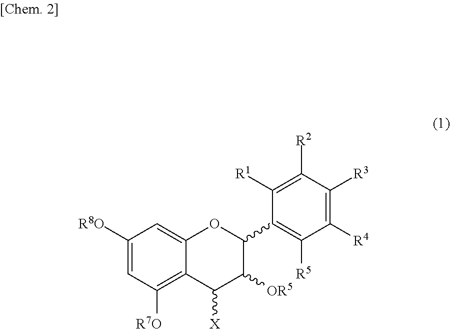

[In general formula (1), R¹ to R⁵ each independently represent a hydrogen atom, a hydroxy group, an alkoxy group, or a substituent represented by OR⁹. R⁶ represents a hydrogen atom, a hydrocarbon group which may have a substituent, an alkoxyalkyl group, an acyl group, a silyl group, or a galloyl group. R⁷ to R⁸ each independently represent a hydrogen atom or a substituent represented by R⁹. R⁹ represents a hydrocarbon group which may have a substituent, an alkoxyalkyl group, an acyl group, or a silyl group. X represents a hydrocarbon group which may have a substituent, a halogen atom, or a substituent in which at least one heteroatom selected from the group consisting of N, O, and S are bonded to a ring-forming atom of the C ring. The wavy line is a single bond indicating an R-configuration or S-configuration.]

The oligomer of the starting flavan derivative is more preferably a compound represented by the following general formula (2).

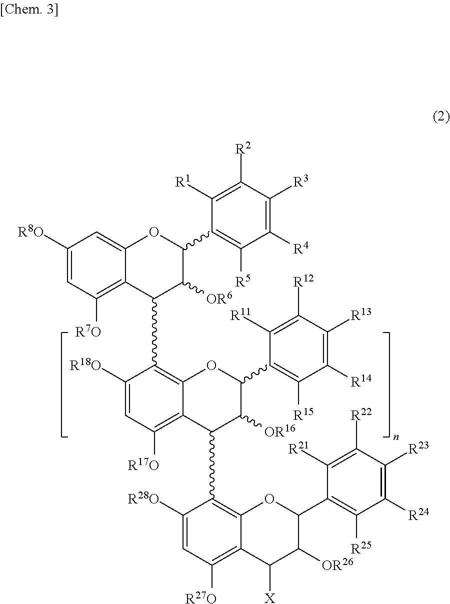

[In general formula (2), R¹ to R⁵, R¹¹ to R¹⁵, and R²¹ to R²⁵ each independently represent a hydrogen atom, a hydroxy group, an alkoxy group, or a substituent represented by OR⁹. R⁶, R¹⁶, and R²⁶ represent a hydrogen atom, a hydrocarbon group which may have a substituent, an alkoxyalkyl group, an acyl group, a silyl group, or a galloyl group. R⁷ to R⁸, R¹¹ to R¹⁸, and R²⁷ to R²⁸ each independently represent a hydrogen atom or a substituent represented by R⁹. R⁹ represents a hydrocarbon group which may have a substituent, an alkoxyalkyl group, an acyl group, or a silyl group. X represents a hydrocarbon group which may have a substituent, a halogen atom, or a substituent in which at least one heteroatom selected from the group consisting of N, O, and S are bonded to a ring-forming atom of the C ring. The wavy line is a single bond indicating an R-configuration or S-configuration. n represents an integer of 0 or larger than 0.]

In general formulas (1) and (2), examples of the alkoxy group include a methoxy group, an ethoxy group, a propoxy group, a butoxy group, and a pentyloxy group. The alkoxy group is preferably a methoxy group.

The hydrocarbon group may be either cyclic or acyclic. It may also be saturated or unsaturated. The hydrocarbon group includes any of a linear aliphatic hydrocarbon group in which carbons are bonded in a straight chain, a branched aliphatic hydrocarbon group in which carbons are branched and bonded, and an aromatic hydrocarbon group. Examples of the hydrocarbon group include an alkyl group, an alkenyl group, an alkynyl group, a dienyl group, a cycloalkyl group, a cycloalkenyl group, an aryl group, and an arylalkyl group.

Examples of the alkyl group include a methyl group, an ethyl group, an n-propyl group, an isopropyl group, an n-butyl group, an isobutyl group, a sec-butyl group, a tert-butyl group, a pentyl group, and a hexyl group. The alkyl group preferably has 1 to 10 carbon atoms, more preferably 1 to 6 carbon atoms, and still more preferably 1 to 4 carbon atoms.

Examples of the alkenyl group include a vinyl group, an allyl group, an isopropenyl group, a 1-butenyl group, a 2-butenyl group, a 1-methyl-2-propenyl group, a 2-methyl-1-propenyl group, a 2-methyl-2-propenyl group, a pentenyl group, and a hexenyl group. The alkenyl group preferably has 2 to 10 carbon atoms, more preferably 2 to 6 carbon atoms, and still more preferably 2 to 4 carbon atoms.

Examples of the alkynyl group include an ethynyl group, a 1-propynyl group, a 2-propynyl group, a 1-butynyl group, a 2-butynyl group, a 3-butynyl group, a 1-methyl-2-propynyl group, a pentynyl group, and a hexynyl group. The alkynyl group preferably has 2 to 10 carbon atoms, more preferably 2 to 6 carbon atoms, and still more preferably 2 to 4 carbon atoms.

Examples of the dienyl group include a 1,3-butadienyl group, a 1,3-pentadienyl group, and a 2,4-pentadienyl group. The dienyl group preferably has 4 to 10 carbon atoms, more preferably 4 to 6 carbon atoms.

Examples of the cycloalkyl group include a cyclopropyl group, a cyclobutyl group, a cyclopentyl group, and a cyclohexyl group. The cycloalkyl group preferably has 3 to 10 carbon atoms, more preferably 4 to 6 carbon atoms.

Examples of the cycloalkenyl group include a cyclopropenyl group, a cyclobutenyl group, a cyclopentenyl group, and a cyclohexenyl group. The cycloalkenyl group preferably has 3 to 10 carbon atoms, more preferably 4 to 6 carbon atoms.

Examples of the aryl group include a phenyl group, a 1-naphthyl group, a 2-naphthyl group, an indenyl group, an o-tolyl group, an m-tolyl group, a p-tolyl group, a 2,3-xylyl group, a 2,4-xylyl group, a 2,5-xylyl group, a 2,6-xylyl group, a 3,4-xylyl group, a 3,5-xylyl group, an o-cumenyl group, an m-cumenyl group, a p-cumenyl group, and a mesityl group. The aryl group preferably has 6 to 10 carbon atoms.

Examples of the arylalkyl group include a benzyl group, a phenethyl group, a 3-phenylpropyl group, a 4-phenylbutyl group, and a triphenylmethyl group. The arylalkyl group preferably has 7 to 10 carbon atoms.

Examples of the alkoxyalkyl group include a methoxymethyl group, an ethoxymethyl group, a propoxymethyl group, a butoxymethyl group, a methoxyethyl group, an ethoxyethyl group, a propoxyethyl group, and a butoxyethyl group. The alkoxyalkyl group preferably has 2 to 6 carbon atoms, more preferably 2 to 4 carbon atoms.

Examples of the acyl group include an acetyl group, a propionyl group, a butyryl group, a benzoyl group, and a naphthoyl group. The acyl group of R⁶, R¹⁶, and R²⁶ is particularly preferably an acetyl group from the viewpoint of the reactivity of activating the flavan derivative.

Examples of the silyl group include a methylsilyl group, an ethylsilyl group, a dimethylsilyl group, a diethylsilyl group, a trimethylsilyl group, a triethylsilyl group, a triiso-propylsilyl group, a tri-tert-butylsilyl group, a dimethyl-tert-butylsilyl group, a trimethoxysilyl group, a triethoxysilyl group, a diphenylmethylsilyl group, a diphenylethylsilyl group, a diphenyliso-propylsilyl group, a diphenyl-tert-butylsilyl group, a triphenylsilyl group, a triphenoxysilyl group, a dimethylmethoxysilyl group, a dimethylphenoxysilyl group, and a methylmethoxyphenylsilyl group.

The substituent in which at least one heteroatom selected from the group consisting of N, O, and S are bonded to a ring-forming atom of the C ring is preferably a substituent in which an electron withdrawing moiety is bonded to the heteroatom bonded to the ring-forming atom of the C ring. The leaving property of the heteroatom is improved due to the coordinating property of the lone pair of electrons of the heteroatom to the Lewis acid.

Examples of the substituent in which N is bonded to a ring-forming atom of the C ring include an azido group, a nitro group, a carbamoyl group, an alkylamino group, a dialkylamino group, an alkoxyalkylamino group, a dialkoxyalkylamino group, an alkylaminoalkylamino group, a dialkylaminoalkylamino group, an alkylsulfanylalkylamino group, a dialkylsulfanylalkylamino group, an arylamino group, an arylaminoalkylamino group, and a nitrogen-containing heterocyclic group. Examples of the nitrogen-containing heterocyclic group include a pyrrolyl group, a pyrazolyl group, an imidazolyl group, a pyridyl group, a pyrazinyl group, a pyrimidinyl group, a pyridazinyl group, an indolyl group, an adenyl group, and a thymidyl group.

Examples of the substituent in which O is bonded to a ring-forming atom of the C ring include a carboxy group, an alkoxy group, an alkoxyalkoxy group, an alkylaminoalkoxy group, an alkylsulfanylalkoxy group, an aryloxy group, and an aryloxyalkoxy group.

Examples of the substituent in which S is bonded to a ring-forming atom of the C ring include an alkylsulfanyl group, an alkoxysulfanyl group, an alkylaminosulfanyl group, an alkylsulfanylalkylsulfanyl group, an arylsulfanyl group, an alkylsulfonyl group, an alkoxysulfonyl group, an arylsulfonyl group, an aryloxysulfonyl group, an alkylsulfinyl group, an alkoxysulfinyl group, an arylsulfinyl group, and an aryloxysulfinyl group.

The hydrocarbon group, the alkoxyalkyl group, the acyl group, the silyl group, the galloyl group, and the substituent in which at least one heteroatom selected from the group consisting of N, O, and S are bonded to a ring-forming atom of the C ring may further have a substituent. One substituent may be introduced into a substitutable position of these substituents, or multiple substituents may be introduced. When multiple substituents are introduced, the substituents may be the same as or different from each other.

Examples of the substituent introduced into these substituents include the above-mentioned alkyl group, alkenyl group, alkynyl group, dienyl group, cycloalkyl group, cycloalkenyl group, aryl group, arylalkyl group, alkoxy group, alkoxyalkyl group, acyl group, and silyl group, as well as a hydroxy group, an amino group, a cyano group, an azido group, a nitro group, a carbamoyl group, and a halogen atom. Examples of the halogen atom include a chlorine atom, a bromine atom, and an iodine atom.

In general formulas (1) and (2), R⁶, R¹⁶, and R²⁶ are particularly preferably an acetyl group. R⁷ to R⁸, R⁷ to R¹⁸, and R²⁷ to R²⁸ are particularly preferably a benzyl group. X is particularly preferably an ethoxyethyl group. n is preferably 0 or more and 15 or less, more preferably 0 or more and 10 or less, and still more preferably 0 or more and 5 or less.

The starting flavan derivative may be any of a (2R,3R)-configuration, a (2R,3S)-configuration, a (2S,3R)-configuration, and a (2S,3S)-configuration with respect to the flavan-3-ol skeleton. The starting flavan derivative is preferably a derivative having a structure in which the substituent represented by X and the substituents represented by OR⁶, OR¹⁶, and OR²⁶ are arranged in syn (cis) from the viewpoint of reactivity.

As the Lewis acid, a Lewis acid having high solubility in the solvent used or a Lewis acid that is liquid at the reaction temperature can be used. The catalyst liquid containing the Lewis acid can be prepared by dissolving the Lewis acid at a predetermined concentration in an appropriate solvent serving as a reaction solvent.

Examples of the Lewis acid that can be used include boron trifluoride diethyl ether complex (BF₃·Et₂O), boron trichloride (BCl₃), trimethylsilyl trifluoromethanesulfonate (TMSOTf), triethylsilyl trifluoromethanesulfonate (TESOTf), triisopropylsilyl trifluoromethanesulfonate, dimethyl-tert-butylsilyl trifluoromethanesulfonate, triphenylsilyl trifluoromethanesulfonate, and other alkylsilyl perfluoroalkylsulfonates.

As the base, an appropriate compound can be used as long as it neutralizes the Lewis acid after the reaction and does not adversely affect the reaction product. The quenching solution containing the base can be prepared by dissolving the base at a predetermined concentration in an appropriate solvent.

Examples of the base that can be used include triethylamine (Et₃N), N,N-diisopropylethylamine, diazabicycloundecene, diazabicyclononene, diazabicyclooctane, pyridine, 2,6-di-tert-butylpyridine, and tetramethylguanidine. In particular, when the quenching solution containing a base is not introduced into the microreactor, an aqueous solution containing an inorganic base such as sodium bicarbonate (NaHCO₃), sodium carbonate (Na₂CO₃), and potassium carbonate (K₂CO₃), which is not soluble in an organic solvent, can also be used.

As the solvent, an appropriate solvent can be used as long as it dissolves the monomer of the flavan derivative, the oligomer of the flavan derivative, and the Lewis acid, does not inhibit the synthesis reaction, and does not adversely affect the reaction product. As the solvent, a polar solvent is preferably used from the viewpoint of carrying out a polymerization reaction similar to an SN1-type nucleophilic substitution reaction.

Examples of the solvent that can be used include dichloromethane, tetrachloromethane, acetone, acetonitrile, methanol, hexane, benzene, toluene, diethyl ether, diisopropyl ether, dimethyl sulfoxide, dimethylformamide, and N-methylpyrrolidone. As the solvent, one of these may be used, or a mixed solvent in which multiple these are mixed may be used.

By the reaction between monomers of the flavan derivative represented by general formula (1), a dimer of the flavan derivative represented by the following general formula (3) is obtained. Depending on the structure of the monomer used in the reaction and the stereoelectronic effect, a dimer having an anti (trans) configuration or syn (cis) configuration of the flavan unit bonded to the 4-position of the C ring with respect to the substituent bonded to the 3-position of the C ring can be obtained.

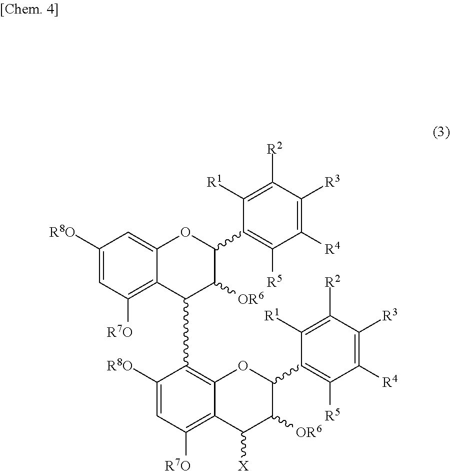

[In general formula (3), R¹ to R⁵, R⁶, R⁷ to R⁹, and X are the same as in general formula (1). The wavy line is a single bond indicating an R-configuration or S-configuration.]

Further, by the reaction between oligomers of the flavan derivative represented by general formula (2), an oligomer of the flavan derivative represented by the following general formula (4) is obtained. Depending on the structure of the oligomer used in the reaction and the stereoelectronic effect, an oligomer having an anti (trans) configuration or syn (cis) configuration of the flavan unit bonded to the 4-position of the C ring with respect to the substituent bonded to the 3-position of the C ring can be obtained. For example, a tetramer is obtained by the reaction between dimers. An octamer is obtained by the reaction between tetramers.

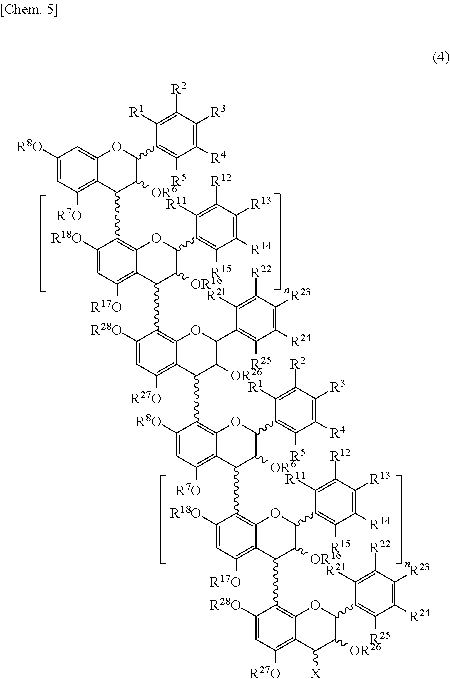

[In general formula (4), R¹ to R⁵, R¹¹ to R¹⁵, R²¹ to R²⁵, R⁶, R¹⁶, R²⁶, R⁷ to R⁹, R¹⁷ to R¹⁸, R²⁷ to R²⁸, X, and n are the same as in general formula (2). The wavy line is a single bond indicating an R-configuration or S-configuration.]

The reaction for producing the oligomer of the flavan derivative is considered to proceed according to the following mechanism. The Lewis acid acts on the flavan derivative to withdraw the electron pair of the substituent (X) bonded to the 4-position carbon of the C ring and is subjected to neighboring group participation by the substituent (OR⁶) bonded to the 3-position carbon of the C ring. A cyclic intermediate is produced as a transition state between the 3-position carbon and the 4-position carbon of the C ring. Then, the substituent (X) is eliminated from the 4-position carbon of the C ring to produce an activated flavan derivative in which the 4-position carbon of the C ring is cationized.

The activated flavan derivative is attacked by an unactivated flavan derivative acting as a nucleophile. Due to the electronic effect, the 8-position carbon of the A ring of the nucleophile becomes a nucleophilic site. By the attack of the nucleophile on the activated form, a bond is formed between the 4-position carbon of the C ring of the activated form and the 8-position carbon of the A ring of the nucleophile. The formation and elimination of the cyclic intermediate are SN1-type regioselective reactions, and regioselective oligomers are produced by stereoelectronic effects.

### First Embodiment

Next, a flavan oligomer production system and a flavan oligomer production method according to the first embodiment of the present invention will be described with reference to the drawings.

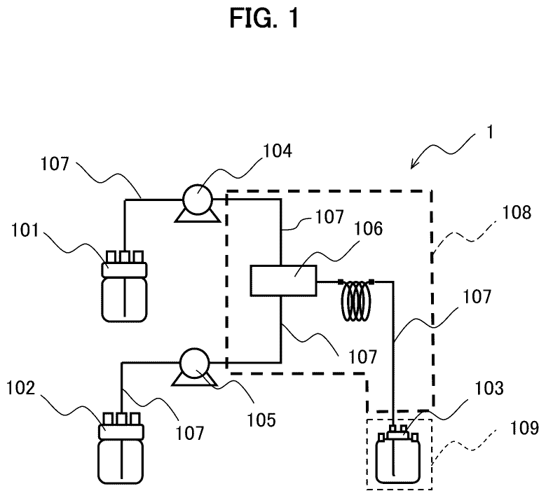

FIG. 1 is a schematic diagram of a flavan oligomer production system according to the first embodiment.

As shown in FIG. 1, the flavan oligomer production system 1 according to the first embodiment includes a raw material liquid container (first container) 101, a catalyst liquid container (second container) 102, a recovery container 103, a first pump 104, a second pump 105, a microreactor 106, a tube 107, a temperature controller 108, a temperature controller 109, and a not-shown fitting for connecting the tube 107 and each component.

As shown by dashed lines in the figure, the temperature controller 108 and the temperature controller 109 are provided so as to adjust a predetermined region in the system to a predetermined temperature. The microreactor 106 and the tube 107 from the microreactor 106 to the recovery container 103 are included in the adjustment range of the temperature controller 108. The recovery container 103 and the tube 107 in the recovery container 103 are included in the adjustment range of the temperature controller 109.

The microreactor 106 is a flow-type reactor and has two inlets through which individual fluids are introduced from the outside, a microchannel for merging the introduced fluids, and an outlet for discharging the product fluid produced by the merging to the outside. The microreactor 106 mixes the fluid introduced from one inlet and the fluid introduced from the other inlet in the microchannel. By mixing the fluids, a product fluid is produced in which a predetermined reaction has begun.

The raw material liquid container 101 and the first pump 104 are connected to one inlet of the microreactor 106 via a tube 107. The raw material liquid container 101 is connected to the suction side of the first pump 104. The discharge side of the first pump 104 is connected to one inlet of the microreactor 106. The raw material liquid container 101 is provided with a raw material liquid containing a flavan derivative as a starting material. The first pump 104 feeds the raw material liquid from the raw material liquid container 101 to one inlet of the microreactor 106.

The catalyst liquid container 102 and the second pump 105 are connected to the other inlet of the microreactor 106 via a tube 107. The catalyst liquid container 102 is connected to the suction side of the second pump 105. The discharge side of the second pump 105 is connected to the other inlet of the microreactor 106. The catalyst liquid container 102 is provided with a catalyst liquid containing a Lewis acid. The second pump 105 feeds the catalyst liquid from the catalyst liquid container 102 to the other inlet of the microreactor 106.

The recovery container 103 is connected to the outlet of the microreactor 106 via a tube 107. The recovery container 103 is a container for recovering the product fluid produced in the microreactor 106 and the subsequent tube 107. A quenching solution containing a base can be stored in the recovery container 103 in order to neutralize the Lewis acid contained in the product fluid.

Examples of the first pump 104 and the second pump 105 include a syringe pump, a tube pump, a plunger pump, a diaphragm pump, and a screw pump, and manual liquid feeding using a syringe, liquid feeding using a head difference, or the like can be used. When a syringe pump is used as the first pump 104 or the second pump 105, a syringe provided with a raw material liquid or a catalyst liquid can be used as a functional alternative to the raw material liquid container 101 or the catalyst liquid container 102, respectively.

As long as they do not adversely affect the raw material liquid, the catalyst liquid, or the product fluid and are not easily deteriorated by them, appropriate materials can be used according to the type of fluid for the material of the microreactor 106, the material of the raw material liquid container 101, the material of the catalyst liquid container 102, the material of the recovery container 103, the material of the tube 107, the material of the tube, syringe, diaphragm, etc. constituting the liquid contact part of the pump, and the material of the fitting.

The material of the microreactor 106, the material of the raw material liquid container 101, the material of the catalyst liquid container 102, the material of the recovery container 103, the material of the tube 107, the material constituting the liquid contact part of the pump, and the material of the fitting may be the same as or different from each other for each installation location in the system. These materials can be appropriately selected according to workability, flexibility, and the like.

Examples of the material of the microreactor 106 include stainless steel, gold, glass, Hastelloy, ceramic, PE (polyethylene), PP (polypropylene), TPX (polymethylpentene), PDMS (polydimethylsiloxane), PC (polycarbonate), and fluorine-based resins such as PTFE (polytetrafluoroethylene) and PFA (perfluoroalkoxyalkane).

The material of the microreactor 106 may be lined with glass or the like, coated with nickel, gold, or the like, or an oxide film such as that formed by oxidation of silicon may be formed to improve corrosion resistance, chemical resistance, and the like.

As the temperature controllers 108 and 109, an appropriate device such as a heat exchanger using a heat medium, a constant temperature water bath using a heat medium, a Peltier temperature controller, or a mantle heater can be used. As the heat medium, water, ethylene glycol, a water/ethylene glycol mixed solvent, dry ice and a water/ethanol mixed solvent, dry ice and a water/methanol mixed solvent, or the like can be used.

The temperature controller 108 and the temperature controller 109 may be adjusted to the same temperature range or different temperature ranges. The temperature by the temperature controllers 108 and 109 can be adjusted according to the reaction rate of the synthesis reaction, the stability of the compound, and the like. Note that the temperature controllers 108 and 109 may not be provided when the synthesis reaction is performed at room temperature or the like.

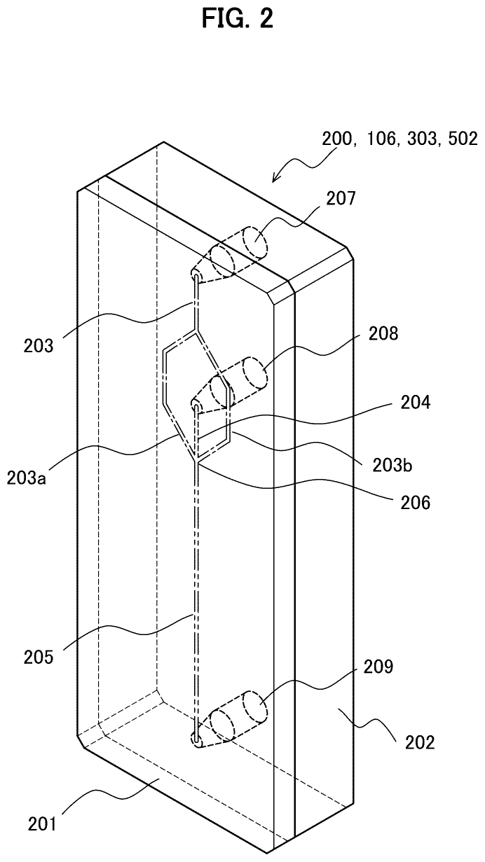

FIG. 2 is a diagram showing an example of a microreactor.

As shown in FIG. 2, a microreactor 200 capable of mixing fluids at different flow rates can also be used as the microreactor used for the synthesis of flavan oligomers.

The microreactor 200 capable of mixing fluids at different flow rates has two inlets (207, 208) through which individual fluids are introduced from the outside, microchannels (203, 204, 205) for merging the introduced fluids, and a fluid outlet 209 for discharging the product fluid produced by the merging at the merging point 206 to the outside.

The microreactor 200 is formed of an upper plate 201 and a lower plate 202. The upper plate 201 is grooved, and the lower plate 202 is stacked so as to cover the grooves, thereby forming microchannels (203, 204, 205). Such grooves can be formed in either the upper plate 201 or the lower plate 202.

The lower plate 202 is provided with through holes (207, 208, 209) at positions overlapping the ends of the microchannels (203, 204, 205). As the through holes (207, 208, 209), a high flow rate side fluid inlet 207, a low flow rate side fluid inlet 208, and a fluid outlet 209 penetrate from the microchannel (203, 204, 205) side to the surface of the lower plate 202 opposite to the microchannel (203, 204, 205).

A not-shown screw groove can be formed in the through holes (207, 208, 209). The tube 107 can be connected via a fitting that can be screwed into the screw groove. Alternatively, the tube 107 can be directly connected to the through holes (207, 208, 209).

The microchannels (203, 204, 205) are constituted by a high flow rate side flow path 203 extending from the high flow rate side fluid inlet 207 to the merging point 206, a low flow rate side flow path 204 extending from the low flow rate side fluid inlet 208 to the merging point 206, and a mixed flow path 205 extending from the merging point 206 to the fluid outlet 209.

The high flow rate side flow path 203 is used to flow a fluid that has a high mixing ratio and is set to a relatively high flow rate among the fluids to be mixed in the microreactor 200. On the other hand, the low flow rate side flow path 204 is used to flow a fluid that has a low mixing ratio and is set to a relatively low flow rate among the fluids to be mixed in the microreactor 200.

In the microreactor 200, the high flow rate side fluid is introduced from the high flow rate side fluid inlet 207, passes through the high flow rate side flow path 203, and reaches the merging point 206. The low flow rate side fluid is introduced from the low flow rate side fluid inlet 208, passes through the low flow rate side flow path 204, and reaches the merging point 206. The high flow rate side fluid and the low flow rate side fluid merge at the merging point 206 to initiate mixing and reaction. These fluids are discharged to the outside from the fluid outlet 209 via the mixed flow path 205.

The high flow rate side flow path 203 is provided with a larger total flow path volume than the low flow rate side flow path 204. For example, the flow path length of the high flow rate side flow path 203 is set longer than that of the low flow rate side flow path 204 provided with the same flow path width and flow path depth. According to such a structure, it is possible to reduce a difference in the timing at which each fluid reaches the merging point 206 when the mixing ratio is biased toward one fluid side and the fluids are controlled to have greatly different flow rates.

The high flow rate side flow path 203 branches into two symmetrical branch flow paths 203a and 203b at an intermediate portion and merges with each other at the merging point 206. The low flow rate side flow path 204 is connected to the merging point 206 from between the two branch flow paths 203a and 203b. At the merging point 206, the low flow rate side fluid and the high flow rate side fluid flowing in from the upstream side flow to the mixed flow path 205 on the downstream side. According to such a structure, the low flow rate side fluid merges with the high flow rate side fluid in a state of being sandwiched between the high flow rate side fluids and initiates mixing. Since the low flow rate side fluid is sandwiched between the high flow rate side fluids, the area of the interface between the fluids is expanded, so that the mixing efficiency can be improved.

It is preferable that the flow path diameter, flow path width, or flow path depth of the high flow rate side flow path 203, the low flow rate side flow path 204, and the mixed flow path 205 be set to 2 mm or less. With such flow paths, effects of the micro reaction field, such as improvement of the surface effect and heat transfer coefficient, can be sufficiently obtained. In particular, it is preferable that the flow path diameter, flow path width, or flow path depth of the high flow rate side flow path 203 and the low flow rate side flow path 204 immediately before the merging point 206, the merging point 206, and the mixed flow path 205 be set to 10 m or more and 1 mm or less. With such flow paths, the fluids can be mixed uniformly and rapidly by molecular diffusion.

Note that the microreactor 200 capable of mixing fluids at different flow rates can also be used when the flow rate ratio of the fluids is 1:1. The mixing of the fluids may be in a form in which the fluids are uniformly mixed, or in a form in which the fluids are non-uniformly mixed, for example, in a form in which multiple phases such as an emulsified state are formed.

In FIG. 2, the microreactor 200 includes the high flow rate side flow path 203 and the low flow rate side flow path 204 having a predetermined shape. However, the microreactor used for the synthesis of flavan oligomers can be provided in an appropriate shape as long as it has a microchannel for mixing at least two fluids. For example, the microchannel may be Y-shaped, T-shaped, or shaped to merge by forming a multilayer flow.

The flow path volumes of the microreactor used for the synthesis of flavan oligomers up to the merging point of the two fluids may be different from each other or may be equivalent to each other. The flow path of the microreactor used for the synthesis of flavan oligomers does not necessarily need to be all microchannels. The flow path diameter, flow path width, or flow path depth of the flow path of the microreactor can be changed according to the type of reaction and the like.

Next, a method for producing flavan oligomers using the flavan oligomer production system 1 will be described.

In the flavan oligomer production system 1, the raw material liquid and the catalyst liquid are mixed in the microreactor 106 to initiate activation of a part of the flavan derivative contained in the raw material liquid by the Lewis acid contained in the catalyst liquid. By the mixing, a product fluid is produced in which a reaction has begun between the part of the flavan derivative activated by the Lewis acid and the remaining flavan derivative acting as a nucleophile. The flavan derivatives can be regioselectively condensed with each other by an SN1-type reaction between the activated flavan derivative and the unactivated flavan derivative, which is the nucleophile.

When producing a flavan oligomer using the production system 1, a raw material liquid containing a flavan derivative having a flavan skeleton is prepared in the raw material liquid container 101. A catalyst liquid containing a Lewis acid is prepared in the catalyst liquid container 102. A quenching solution containing a base is prepared in the recovery container 103.

First, the raw material liquid containing the flavan derivative prepared in the raw material liquid container 101 is fed from the raw material liquid container 101 to one inlet of the microreactor 106 by the first pump 104. Further, the catalyst liquid containing the Lewis acid prepared in the catalyst liquid container 102 is fed from the catalyst liquid container 102 to the other inlet of the microreactor 106 by the second pump 105.

Next, the raw material liquid containing the flavan derivative and the catalyst liquid containing the Lewis acid are mixed in the microreactor 106. By the mixing, activation of a part of the flavan derivative contained in the raw material liquid by the Lewis acid contained in the catalyst liquid is initiated. Then, a reaction between the activated part of the flavan derivative and the remaining unactivated flavan derivative acting as a nucleophile is initiated. The reaction between the flavan derivatives further proceeds while the product fluid produced in the microreactor 106 flows in the subsequent tube 107 toward the downstream.

In the microreactor 106 of the production system 1, the flavan derivative, which is a starting material, and the Lewis acid are reacted at a reaction equivalent ratio close to flavan derivative:Lewis acid=1:0.5. Therefore, the flow rate ratio of the raw material liquid and the catalyst liquid introduced into the microreactor 106, the concentration of the raw material liquid prepared in the raw material liquid container 101, and the concentration of the catalyst liquid prepared in the catalyst liquid container 102 are adjusted so as to achieve such a reaction equivalent ratio. The reaction equivalent ratio of the flavan derivative and the Lewis acid may be adjusted only by the flow rate ratio, may be adjusted only by the concentration, or may be adjusted by both the flow rate ratio and the concentration.

Subsequently, the product fluid discharged from the microreactor 106 and the subsequent tube 107 is recovered into the quenching solution containing a base in the recovery container 103. By recovering into the quenching solution, the Lewis acid in the product fluid is neutralized with the base to terminate the polymerization reaction. When the reaction is initiated in the microchannel of the microreactor 106, the reaction proceeds through the subsequent tube 107, and the product fluid is recovered into the quenching solution containing a base to terminate the polymerization reaction, an oligomer is obtained in which the flavan derivatives are bonded at a desired degree of polymerization.

The produced oligomer of the flavan derivative can be deprotected by removing the protecting group after separation and purification, if necessary. The oligomer of the flavan derivative can be subjected to a step of introducing a new substituent, a step of introducing a modified structure such as a sugar chain, other reaction steps of converting a three-dimensional structure, a skeleton, a functional group, or the like, and the like before or after the deprotection.

According to the flavan oligomer production system 1 and the flavan oligomer production method described above, the flavan derivative contained in the raw material liquid and the Lewis acid contained in the catalyst liquid can efficiently react in the microchannel of the microreactor 106 and the subsequent tube 107. Therefore, only a part of the flavan derivative contained in the raw material liquid can be activated by the Lewis acid at a predetermined reaction equivalent ratio. The activated part of the flavan derivative and the remaining flavan derivative acting as a nucleophile can be reacted at a predetermined reaction equivalent ratio. By using a microreactor, it is possible to precisely control the flow rate ratio of the fluids, the reaction ratio of the reactants, the initiation time of the reaction, and the termination time of the reaction. Therefore, a flavan oligomer in which flavan derivatives are bonded at a desired degree of polymerization can be efficiently synthesized in high yield.

Further, according to the flavan oligomer production system 1 and the flavan oligomer production method described above, since the yield of the oligomer having the desired degree of polymerization is improved, it is possible to reduce the cost and labor for separating and purifying the oligomer having the desired degree of polymerization from the mixture after the synthesis. Further, an excess amount of catalyst is not required, and the cost of the catalyst and the purification cost are reduced.

The reaction equivalent ratio of the flavan derivative and the Lewis acid to be reacted in the microreactor 106 of the production system 1 and the subsequent tube 107 is not particularly limited as long as the Lewis acid is 0.5 equivalent or more with respect to 1 equivalent of the flavan derivative. The reaction equivalent ratio of the flavan derivative and the Lewis acid depends on the types of the flavan derivative and the Lewis acid, but from the viewpoint of saving the amounts of the flavan derivative and the Lewis acid used, the ratio is preferably flavan derivative:Lewis acid=1:0.5 to 1:1. The reaction equivalent ratio of the Lewis acid is preferably 1 equivalent or less, more preferably 0.5 equivalent or more and 0.9 equivalent or less, still more preferably 0.5 equivalent or more and 0.8 equivalent or less, further preferably 0.5 equivalent or more and 0.7 equivalent or less, and still further preferably 0.5 equivalent or more and 0.6 equivalent or less with respect to 1 equivalent of the flavan derivative.

With such a reaction equivalent ratio, the part of the flavan derivative activated by the Lewis acid and the remaining flavan derivative acting as a nucleophile are close to a reaction equivalent ratio of 1:1. Since the flavan derivatives can be reacted with each other at a ratio of 1:1, unreacted flavan derivatives (monomers and the like) can be reduced, and since the reaction conditions become more consistent by using the microreactor 106, oligomers having a degree of polymerization equal to or higher than the desired degree of polymerization can be reduced. That is, a dimer having a desired degree of polymerization or an oligomer having a desired degree of polymerization can be obtained in high yield using a monomer of the flavan derivative or an oligomer of the flavan derivative as a starting material.

The reaction equivalent ratio of the Lewis acid and the base to be reacted in the recovery container 103 can be set to an appropriate reaction equivalent ratio as long as the multimerization reaction of the flavan derivative is terminated. The reaction equivalent ratio of the base is preferably 1 equivalent or more, more preferably an excess amount exceeding 1 equivalent, with respect to 1 equivalent of the Lewis acid to be reacted in the microreactor 106. With such an amount, the reaction can be safely terminated even if the Lewis acid does not react properly. The reaction equivalent ratio of the base may be a large excess amount when the quenching solution is stored in the recovery container 103, when separation and purification are planned, or the like.

### Second Embodiment

Next, a flavan oligomer production system and a flavan oligomer production method according to the second embodiment of the present invention will be described with reference to the drawings.

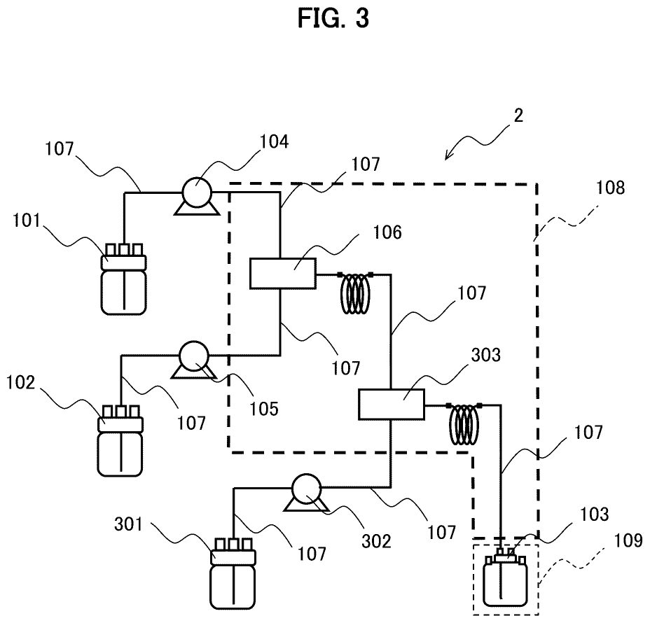

FIG. 3 is a schematic diagram of a flavan oligomer production system according to the second embodiment.

As shown in FIG. 3, the flavan oligomer production system 2 according to the second embodiment includes a raw material liquid container (first container) 101, a catalyst liquid container (second container) 102, a recovery container 103, a raw material liquid container (third container) 301, a first pump 104, a second pump 105, a third pump 302, a first microreactor 106, a second microreactor 303, a tube 107, a temperature controller 108, a temperature controller 109, and a not-shown fitting for connecting the tube 107 and each component.

The production system 2 is different from the production system 1 in that it includes multiple stages of microreactors 106 and 303 connected in series with each other, and the flavan oligomer synthesis reaction is performed stepwise.

In the production system 2, the second microreactor 303 is connected downstream of the first microreactor 106. Further, the raw material liquid container 301 and the third pump 302 are connected to the second microreactor 303.

As shown by dashed lines in the figure, the temperature controller 108 and the temperature controller 109 are provided so as to adjust a predetermined region in the system to a predetermined temperature. The first microreactor 106, the tube 107 from the first microreactor 106 to the second microreactor 303, the second microreactor 303, and the tube 107 from the second microreactor 303 to the recovery container 103 are included in the adjustment range of the temperature controller 108. The recovery container 103 and the tube 107 in the recovery container 103 are included in the adjustment range of the temperature controller 109.

The first microreactor 106 and the second microreactor 303 are flow-type reactors and have two inlets through which individual fluids are introduced from the outside, a microchannel for merging the introduced fluids, and an outlet for discharging the product fluid produced by the merging to the outside. These microreactors 106 and 303 mix the fluid introduced from one inlet and the fluid introduced from the other inlet in the microchannel. By mixing the fluids, a product fluid is produced in which a predetermined reaction has begun.

In the production system 2, the raw material liquid container 101 and the first pump 104 are connected to one inlet of the first microreactor 106 via a tube 107, as in the production system 1. The catalyst liquid container 102 and the second pump 105 are connected to the other inlet of the first microreactor 106 via a tube 107, as in the production system 1.

One inlet of the second microreactor 303 is connected to the outlet of the first microreactor 106 via a tube 107. A first product fluid produced in the first microreactor 106 and the subsequent tube 107 is fed to the second microreactor 303.

The raw material liquid container 301 and the third pump 302 are connected to the other inlet of the second microreactor 303 via a tube 107. The raw material liquid container 301 is connected to the suction side of the third pump 302. The discharge side of the third pump 302 is connected to the other inlet of the second microreactor 303. The raw material liquid container 301 is provided with a raw material liquid containing a flavan derivative as a starting material. The third pump 302 feeds the raw material liquid from the raw material liquid container 301 to the other inlet of the second microreactor 303.

The recovery container 103 is connected to the outlet of the second microreactor 303 via a tube 107. The recovery container 103 is a container for recovering a second product fluid produced in the second microreactor 303 and the subsequent tube 107. A quenching solution containing a base can be stored in the recovery container 103 in order to neutralize the Lewis acid contained in the second product fluid.

As the first pump 104 and the second pump 105, an appropriate pump can be used as in the production system 1. As the third pump 302, an appropriate pump can be used as in the first pump 104 and the second pump 105. When a syringe pump is used as the first pump 104, the second pump 105, or the third pump 302, a syringe provided with a raw material liquid or a catalyst liquid can be used as a functional alternative to the raw material liquid container 101, the catalyst liquid container 102, or the raw material liquid container 301, respectively.

As with the production system 1, appropriate materials can be used for the material of the first microreactor 106, the material of the second microreactor 303, the material of the raw material liquid container 101, the material of the catalyst liquid container 102, the material of the recovery container 103, the material of the raw material liquid container 301, the material of the tube 107, the material of the tube, syringe, diaphragm, etc. constituting the liquid contact part of the pump, and the material of the fitting.

The microreactor 200 (see FIG. 2) capable of mixing fluids at different flow rates can be used as the first microreactor 106 and the second microreactor 303. The microreactor 200 capable of mixing fluids at different flow rates may be used only for the first microreactor 106 or only for the second microreactor 303, but is preferably used for both the first microreactor 106 and the second microreactor 303.

Next, a method for producing flavan oligomers using the flavan oligomer production system 2 will be described.

In the flavan oligomer production system 2, the raw material liquid and the catalyst liquid are mixed in the first microreactor 106 to initiate activation of the flavan derivative contained in the raw material liquid by the Lewis acid contained in the catalyst liquid. By the first mixing, a first product fluid containing the flavan derivative activated by the Lewis acid is produced. Then, the first product fluid and the raw material liquid are mixed in the second microreactor 303 to initiate a reaction between the flavan derivative activated by the Lewis acid and the unactivated flavan derivative acting as a nucleophile. By the second mixing, a second product fluid is produced in which a reaction has begun between the flavan derivatives. The flavan derivatives can be regioselectively condensed with each other by an SN1-type reaction between the activated flavan derivative and the unactivated flavan derivative, which is the nucleophile.

When producing a flavan oligomer using the production system 2, a raw material liquid containing a flavan derivative having a flavan skeleton is prepared in the raw material liquid container 101. A catalyst liquid containing a Lewis acid is prepared in the catalyst liquid container 102. A raw material liquid containing a flavan derivative having a flavan skeleton is prepared in the raw material liquid container 301. A quenching solution containing a base is prepared in the recovery container 103.

First, the raw material liquid containing the flavan derivative prepared in the raw material liquid container 101 is fed from the raw material liquid container 101 to one inlet of the first microreactor 106 by the first pump 104. Further, the catalyst liquid containing the Lewis acid prepared in the catalyst liquid container 102 is fed from the catalyst liquid container 102 to the other inlet of the first microreactor 106 by the second pump 105.

Next, the raw material liquid containing the flavan derivative and the catalyst liquid containing the Lewis acid are mixed in the first microreactor 106. By the mixing, activation of the flavan derivative contained in the raw material liquid by the Lewis acid contained in the catalyst liquid is initiated. The activation of the flavan derivative further proceeds while the first product fluid produced in the first microreactor 106 flows in the subsequent tube 107 toward the downstream. The first product fluid is fed from the first microreactor 106 to one inlet of the second microreactor 303.

In the first microreactor 106 of the production system 2, the flavan derivative, which is a starting material, and the Lewis acid are reacted at a reaction equivalent ratio close to flavan derivative:Lewis acid=1:1. Therefore, the flow rate ratio of the raw material liquid and the catalyst liquid introduced into the first microreactor 106, the concentration of the raw material liquid prepared in the raw material liquid container 101, and the concentration of the catalyst liquid prepared in the catalyst liquid container 102 are adjusted so as to achieve such a reaction equivalent ratio. The reaction equivalent ratio of the flavan derivative and the Lewis acid may be adjusted only by the flow rate ratio, may be adjusted only by the concentration, or may be adjusted by both the flow rate ratio and the concentration.

Subsequently, the raw material liquid containing the flavan derivative prepared in the raw material liquid container 301 is fed from the raw material liquid container 301 to the other inlet of the second microreactor 303 by the third pump 302.

Next, the first product fluid containing the activated flavan derivative and the raw material liquid containing the unactivated flavan derivative acting as a nucleophile are mixed in the second microreactor 303. By the mixing, a reaction between the activated flavan derivative and the unactivated flavan derivative acting as a nucleophile is initiated. The reaction between the flavan derivatives further proceeds while the second product fluid produced in the second microreactor 303 flows in the subsequent tube 107 toward the downstream.

In the second microreactor 303 of the production system 2, the flavan derivative activated in the first microreactor 106 and the subsequent tube 107 and the unactivated flavan derivative prepared in the raw material liquid container 301 are reacted at a reaction equivalent ratio close to activated form:unactivated form=1:1. Therefore, the flow rate ratio of the first product fluid and the raw material liquid introduced into the second microreactor 303 and the concentration of the raw material liquid prepared in the raw material liquid container 301 are adjusted so as to achieve such a reaction equivalent ratio. The reaction equivalent ratio of the activated flavan derivative and the unactivated flavan derivative may be adjusted only by the flow rate ratio, may be adjusted only by the concentration, or may be adjusted by both the flow rate ratio and the concentration.

Subsequently, the reacting second product fluid discharged from the second microreactor 303 and the subsequent tube 107 is recovered into the quenching solution containing a base in the recovery container 103. By recovering into the quenching solution, the Lewis acid in the second product fluid is neutralized with the base to terminate the polymerization reaction. When the polymerization reaction is initiated in the microchannel of the second microreactor 303, the reaction proceeds through the subsequent tube 107, and the product fluid is recovered into the quenching solution containing a base to terminate the polymerization reaction, an oligomer is obtained in which the flavan derivatives are bonded at a desired degree of polymerization.

The produced oligomer of the flavan derivative can be deprotected by removing the protecting group after separation and purification, if necessary. The oligomer of the flavan derivative can be subjected to a step of introducing a new substituent, a step of introducing a modified structure such as a sugar chain, other reaction steps of converting a three-dimensional structure, a skeleton, a functional group, or the like, and the like before or after the deprotection.

According to the flavan oligomer production system 2 and the flavan oligomer production method described above, the flavan derivative contained in the raw material liquid and the Lewis acid contained in the catalyst liquid can efficiently react in the microchannel of the first microreactor 106 and the subsequent tube 107, and a first product fluid is produced. Further, the activated flavan derivative contained in the first product fluid and the flavan derivative contained in the raw material liquid can efficiently react in the microchannel of the second microreactor 303 and the subsequent tube 107. Therefore, the activated flavan derivative and the flavan derivative acting as a nucleophile can be reacted at a predetermined reaction equivalent ratio. By using a microreactor, it is possible to precisely control the flow rate ratio of the fluids, the reaction ratio of the reactants, the initiation time of the reaction, and the termination time of the reaction. Therefore, a flavan oligomer in which flavan derivatives are bonded at a desired degree of polymerization can be efficiently synthesized in high yield.

Further, according to the flavan oligomer production system 2 and the flavan oligomer production method described above, since the yield of the oligomer having the desired degree of polymerization is improved, it is possible to reduce the cost and labor for separating and purifying the oligomer having the desired degree of polymerization from the mixture after the synthesis. Further, an excess amount of catalyst is not required, and the cost of the catalyst and the purification cost are reduced. According to the flavan oligomer production system 2 and the flavan oligomer production method, control of the reaction conditions becomes easier compared to the production system 1.

The reaction equivalent ratio of the flavan derivative and the Lewis acid to be reacted in the first microreactor 106 of the production system 2 and the tube 107 on the downstream side of the first microreactor 106 is not particularly limited as long as the Lewis acid is 1 equivalent or more with respect to 1 equivalent of the flavan derivative. The reaction equivalent ratio of the flavan derivative and the Lewis acid depends on the types of the flavan derivative and the Lewis acid, but from the viewpoint of saving the amount of the Lewis acid used, the ratio is preferably flavan derivative:Lewis acid=1:1 to 1:2. The reaction equivalent ratio of the Lewis acid is preferably 2 equivalents or less, more preferably 1.0 equivalent or more and 1.8 equivalents or less, still more preferably 1.0 equivalent or more and 1.6 equivalents or less, further preferably 1.0 equivalent or more and 1.4 equivalents or less, and still further preferably 1.0 equivalent or more and 1.2 equivalents or less with respect to 1 equivalent of the flavan derivative.

The reaction equivalent ratio of the activated flavan derivative and the flavan derivative acting as a nucleophile to be reacted in the second microreactor 303 of the production system 2 and the tube 107 on the downstream side of the second microreactor 303 is not particularly limited as long as the flavan derivative acting as a nucleophile is 1 equivalent or more with respect to 1 equivalent of the activated flavan derivative. The reaction equivalent ratio of the activated flavan derivative and the flavan derivative acting as a nucleophile depends on the types of the flavan derivative and the Lewis acid, but from the viewpoint of saving the amount of the flavan derivative used, the ratio is preferably activated flavan derivative:flavan derivative acting as a nucleophile=1:1 to 1:2. The reaction equivalent ratio of the flavan derivative acting as a nucleophile is preferably 2 equivalents or less, more preferably 1.0 equivalent or more and 1.8 equivalents or less, still more preferably 1.0 equivalent or more and 1.6 equivalents or less, further preferably 1.0 equivalent or more and 1.4 equivalents or less, and still further preferably 1.0 equivalent or more and 1.2 equivalents or less with respect to 1 equivalent of the activated flavan derivative.

With a reaction equivalent ratio such that the amount of the flavan derivative acting as a nucleophile is excessive, the flavan derivative activated by the Lewis acid and the flavan derivative acting as a nucleophile can be reliably reacted, but unreacted flavan derivatives remain. As the reaction equivalent ratio of the activated flavan derivative and the flavan derivative acting as a nucleophile approaches 1:1, flavan derivatives (monomers and the like) can be reduced, and since the microreactors 106 and 303 are used, the reaction conditions become more consistent, thereby reducing oligomers having a degree of polymerization equal to or higher than the desired degree of polymerization. That is, a dimer having a desired degree of polymerization or an oligomer having a desired degree of polymerization can be obtained in high yield using a monomer of the flavan derivative or an oligomer of the flavan derivative as a starting material.

The reaction equivalent ratio of the Lewis acid and the base to be reacted in the recovery container 103 can be set to the same conditions as in the production system 1.

In the production system 2, the first microreactor 106, the tube 107 from the first microreactor 106 to the second microreactor 303, the second microreactor 303, and the tube 107 from the second microreactor 303 to the recovery container 103 are included in the adjustment range of the temperature controller 108. With such a configuration, the upstream side and the downstream side of the second microreactor 303 can be adjusted to substantially the same temperature. Therefore, cost reduction and simplification of control are possible.

However, the first microreactor 106 and the tube 107 from the first microreactor 106 to the second microreactor 303, and the second microreactor 303 and the tube 107 from the second microreactor 303 to the recovery container 103 can be adjusted by different temperature controllers. More precisely, the upstream side and the downstream side of the second microreactor 303 can be adjusted to different temperatures. Note that in order to rapidly terminate the polymerization reaction, a quenching solution containing an excess amount of a base can be prepared in the recovery container 103, and the temperature controller 109 that adjusts the temperature of the recovery container 103 and the tube 107 in the recovery container 103 can be adjusted to a higher temperature than these sections.

### Third Embodiment

Next, a flavan oligomer production system and a flavan oligomer production method according to the third embodiment of the present invention will be described with reference to the drawings.

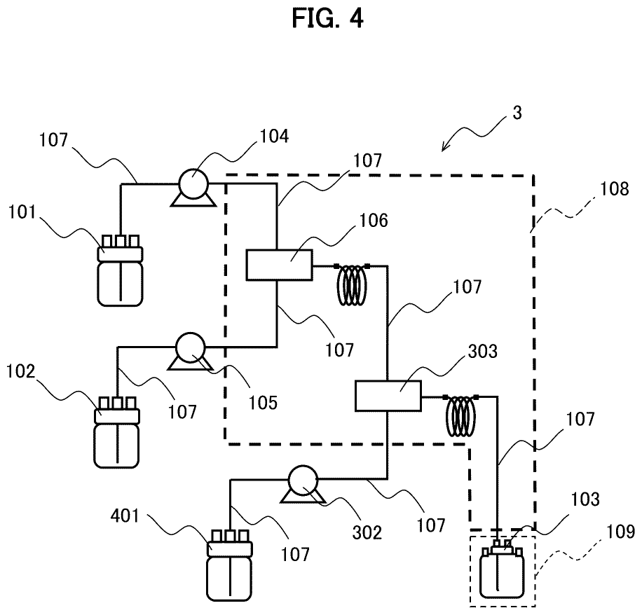

FIG. 4 is a schematic diagram of a flavan oligomer production system according to the third embodiment.

As shown in FIG. 4, the flavan oligomer production system 3 according to the third embodiment includes a raw material liquid container (first container) 101, a catalyst liquid container (second container) 102, a recovery container 103, a quenching solution container (fourth container) 401, a first pump 104, a second pump 105, a third pump 302, a first microreactor 106, a second microreactor 303, a tube 107, a temperature controller 108, a temperature controller 109, and a not-shown fitting for connecting the tube 107 and each component.

The production system 3 is different from the production system 1 in that it includes multiple stages of microreactors 106 and 303 connected in series with each other, and the flavan oligomer synthesis reaction is terminated by mixing a base in the microreactor.

In the production system 3, the second microreactor 303 is connected downstream of the first microreactor 106. The quenching solution container 401 and the third pump 302 are connected to the second microreactor 303.

As shown by dashed lines in the figure, the temperature controller 108 and the temperature controller 109 are provided so as to adjust a predetermined region in the system to a predetermined temperature. The first microreactor 106, the tube 107 from the first microreactor 106 to the second microreactor 303, the second microreactor 303, and the tube 107 from the second microreactor 303 to the recovery container 103 are included in the adjustment range of the temperature controller 108. The recovery container 103 and the tube 107 in the recovery container 103 are included in the adjustment range of the temperature controller 109.

In the production system 3, the raw material liquid container 101 and the first pump 104 are connected to one inlet of the first microreactor 106 via a tube 107, as in the production system 1. The catalyst liquid container 102 and the second pump 105 are connected to the other inlet of the first microreactor 106 via a tube 107, as in the production system 1.

One inlet of the second microreactor 303 is connected to the outlet of the first microreactor 106 via a tube 107. A first product fluid produced in the first microreactor 106 and the subsequent tube 107 is fed to the second microreactor 303.

The quenching solution container 401 and the third pump 302 are connected to the other inlet of the second microreactor 303 via a tube 107. The quenching solution container 401 is connected to the suction side of the third pump 302. The discharge side of the third pump 302 is connected to the other inlet of the second microreactor 303. The quenching solution container 401 is provided with a quenching solution containing a base. The third pump 302 feeds the quenching solution from the quenching solution container 401 to the other inlet of the second microreactor 303.

The recovery container 103 is connected to the outlet of the second microreactor 303 via a tube 107. The recovery container 103 is a container for recovering a second product fluid produced in the second microreactor 303 and the subsequent tube 107. A quenching solution containing a base may or may not be stored in the recovery container 103 in order to neutralize the Lewis acid contained in the second product fluid.

As the first pump 104 and the second pump 105, an appropriate pump can be used as in the production system 1. As the third pump 302, an appropriate pump can be used as in the first pump 104 and the second pump 105. When a syringe pump is used as the first pump 104, the second pump 105, or the third pump 302, a syringe provided with a raw material liquid, a catalyst liquid, or a quenching solution can be used as a functional alternative to the raw material liquid container 101, the catalyst liquid container 102, or the quenching solution container 401, respectively.

As with the production system 1, appropriate materials can be used for the material of the first microreactor 106, the material of the second microreactor 303, the material of the raw material liquid container 101, the material of the catalyst liquid container 102, the material of the recovery container 103, the material of the quenching solution container 401, the material of the tube 107, the material of the tube, syringe, diaphragm, etc. constituting the liquid contact part of the pump, and the material of the fitting.

The microreactor 200 (see FIG. 2) capable of mixing fluids at different flow rates can be used as the first microreactor 106 and the second microreactor 303. The microreactor 200 capable of mixing fluids at different flow rates may be used only for the first microreactor 106 or only for the second microreactor 303, but is preferably used for both the first microreactor 106 and the second microreactor 303.

Next, a method for producing flavan oligomers using the flavan oligomer production system 3 will be described.

In the flavan oligomer production system 3, the raw material liquid and the catalyst liquid are mixed in the first microreactor 106 to initiate activation of a part of the flavan derivative contained in the raw material liquid by the Lewis acid contained in the catalyst liquid. By the first mixing, a first product fluid is produced in which a reaction has begun between the part of the flavan derivative activated by the Lewis acid and the remaining flavan derivative acting as a nucleophile. Then, the first product fluid and the quenching solution are mixed in the second microreactor 303 to initiate a neutralization reaction between the Lewis acid and the base. By the second mixing, a second product fluid is produced in which termination of the reaction between the flavan derivatives has begun. The flavan derivatives can be regioselectively condensed with each other by an SN1-type reaction between the activated flavan derivative and the unactivated flavan derivative, which is the nucleophile. Thereafter, the unintended polymerization reaction can be forcibly terminated by the reaction between the Lewis acid and the base.

When producing a flavan oligomer using the production system 3, a raw material liquid containing a flavan derivative having a flavan skeleton is prepared in the raw material liquid container 101. A catalyst liquid containing a Lewis acid is prepared in the catalyst liquid container 102. A quenching solution containing a base is prepared in the quenching solution container 401. A quenching solution containing a base may or may not be prepared in the recovery container 103.

First, the raw material liquid containing the flavan derivative prepared in the raw material liquid container 101 is fed from the raw material liquid container 101 to one inlet of the first microreactor 106 by the first pump 104. Further, the catalyst liquid containing the Lewis acid prepared in the catalyst liquid container 102 is fed from the catalyst liquid container 102 to the other inlet of the first microreactor 106 by the second pump 105.

Next, the raw material liquid containing the flavan derivative and the catalyst liquid containing the Lewis acid are mixed in the first microreactor 106. By the mixing, activation of a part of the flavan derivative contained in the raw material liquid by the Lewis acid contained in the catalyst liquid is initiated. Then, a reaction between the activated part of the flavan derivative and the remaining unactivated flavan derivative acting as a nucleophile is initiated. The reaction between the flavan derivatives further proceeds while the first product fluid produced in the first microreactor 106 flows in the subsequent tube 107 toward the downstream. The first product fluid is fed from the first microreactor 106 to one inlet of the second microreactor 303.

In the first microreactor 106 of the production system 3, the flavan derivative, which is a starting material, and the Lewis acid are reacted at a reaction equivalent ratio close to flavan derivative:Lewis acid=1:0.5. Therefore, the flow rate ratio of the raw material liquid and the catalyst liquid introduced into the first microreactor 106, the concentration of the raw material liquid prepared in the raw material liquid container 101, and the concentration of the catalyst liquid prepared in the catalyst liquid container 102 are adjusted so as to achieve such a reaction equivalent ratio. The reaction equivalent ratio of the flavan derivative and the Lewis acid may be adjusted only by the flow rate ratio, may be adjusted only by the concentration, or may be adjusted by both the flow rate ratio and the concentration.

Subsequently, the quenching solution containing the base prepared in the quenching solution container 401 is fed from the quenching solution container 401 to the other inlet of the second microreactor 303 by the third pump 302.

Next, the first product fluid in which the polymerization reaction is in progress and the quenching solution containing the base are mixed in the second microreactor 303. By the mixing, a neutralization reaction between the Lewis acid and the base is initiated. The neutralization reaction further proceeds while the second product fluid produced in the second microreactor 303 flows in the subsequent tube 107 toward the downstream. By the neutralization, the reaction between the unactivated flavan derivative and the Lewis acid and the reaction between the oligomer of the flavan derivative produced in the polymerization reaction and the Lewis acid are terminated.

In the second microreactor 303 of the production system 3, the Lewis acid in the first product fluid produced in the first microreactor 106 and the subsequent tube 107 and the base prepared in the quenching solution container 401 are reacted so that the base is 1 equivalent or more with respect to 1 equivalent of the Lewis acid. Therefore, the flow rate ratio of the first product fluid and the quenching solution introduced into the second microreactor 303 and the concentration of the quenching solution prepared in the quenching solution container 401 are adjusted so as to achieve such a reaction equivalent ratio. The reaction equivalent ratio of the Lewis acid and the base may be adjusted only by the flow rate ratio, may be adjusted only by the concentration, or may be adjusted by both the flow rate ratio and the concentration.

Subsequently, the second product fluid discharged from the second microreactor 303 and the subsequent tube 107 is recovered into the recovery container 103. When the polymerization reaction is initiated in the microchannel of the first microreactor 106, the reaction proceeds through the subsequent tube 107, the polymerization reaction termination is initiated in the microchannel of the second microreactor 303, and the reaction proceeds through the subsequent tube 107, an oligomer is obtained in which the flavan derivatives are bonded at a desired degree of polymerization.

The produced oligomer of the flavan derivative can be deprotected by removing the protecting group after separation and purification, if necessary. The oligomer of the flavan derivative can be subjected to a step of introducing a new substituent, a step of introducing a modified structure such as a sugar chain, other reaction steps of converting a three-dimensional structure, a skeleton, a functional group, or the like, and the like before or after the deprotection.

According to the flavan oligomer production system 3 and the flavan oligomer production method described above, the flavan derivative contained in the raw material liquid and the Lewis acid contained in the catalyst liquid can efficiently react in the microchannel of the first microreactor 106 and the subsequent tube 107, and a first product fluid is produced. Further, the Lewis acid contained in the first product fluid and the base contained in the quenching solution can efficiently react in the microchannel of the second microreactor 303 and the subsequent tube 107. Therefore, the activated flavan derivative and the flavan derivative acting as a nucleophile can be reacted at a predetermined reaction equivalent ratio, and then the Lewis acid can be neutralized to terminate the multimerization reaction of the flavan derivative. By using a microreactor, it is possible to precisely control the flow rate ratio of the fluids, the reaction ratio of the reactants, the initiation time of the reaction, and the termination time of the reaction. Therefore, a flavan oligomer in which flavan derivatives are bonded at a desired degree of polymerization can be efficiently synthesized in high yield.

Further, according to the flavan oligomer production system 3 and the flavan oligomer production method described above, since the yield of the oligomer having the desired degree of polymerization is improved, it is possible to reduce the cost and labor for separating and purifying the oligomer having the desired degree of polymerization from the mixture after the synthesis. Further, an excess amount of catalyst is not required, and the cost of the catalyst and the purification cost are reduced. According to the flavan oligomer production system 3 and the flavan oligomer production method, the reaction can be terminated rapidly and reliably compared to the production system 1.

The reaction equivalent ratio of the flavan derivative and the Lewis acid to be reacted in the first microreactor 106 of the production system 3 and the tube 107 on the downstream side of the first microreactor 106 can be set to the same conditions as in the production system 1. The reaction equivalent ratio of the flavan derivative and the Lewis acid depends on the types of the flavan derivative and the Lewis acid, but the ratio is preferably flavan derivative:Lewis acid=1:0.5 to 1:1.

The reaction equivalent ratio of the Lewis acid and the base to be reacted in the second microreactor 303 of the production system 3 and the tube 107 on the downstream side of the second microreactor 303 can be set to an appropriate reaction equivalent ratio as long as the multimerization reaction of the flavan derivative is terminated. The reaction equivalent ratio of the base is preferably 1 equivalent or more, more preferably an excess amount exceeding 1 equivalent, with respect to 1 equivalent of the Lewis acid to be reacted in the first microreactor 106. With such an amount, the reaction can be safely terminated even if the Lewis acid does not react properly. The reaction equivalent ratio of the base can be set to an amount in consideration of the amount of the quenching solution in the recovery container 103 when the quenching solution is stored in the recovery container 103, and may be a large excess amount when separation and purification are planned.

In the production system 3, the first microreactor 106, the tube 107 from the first microreactor 106 to the second microreactor 303, the second microreactor 303, and the tube 107 from the second microreactor 303 to the recovery container 103 are included in the adjustment range of the temperature controller 108. With such a configuration, the upstream side and the downstream side of the second microreactor 303 can be adjusted to substantially the same temperature. Therefore, cost reduction and simplification of control are possible.

However, the first microreactor 106 and the tube 107 from the first microreactor 106 to the second microreactor 303, and the second microreactor 303 and the tube 107 from the second microreactor 303 to the recovery container 103 can be adjusted by different temperature controllers. More precisely, the upstream side and the downstream side of the second microreactor 303 can be adjusted to different temperatures. Note that in order to reliably terminate the polymerization reaction, a quenching solution containing an excess amount of a base can be prepared in the recovery container 103, and the temperature controller 109 that adjusts the temperature of the recovery container 103 and the tube 107 in the recovery container 103 can be adjusted to a higher temperature than these sections.

### Fourth Embodiment

Next, a flavan oligomer production system and a flavan oligomer production method according to the fourth embodiment of the present invention will be described with reference to the drawings.

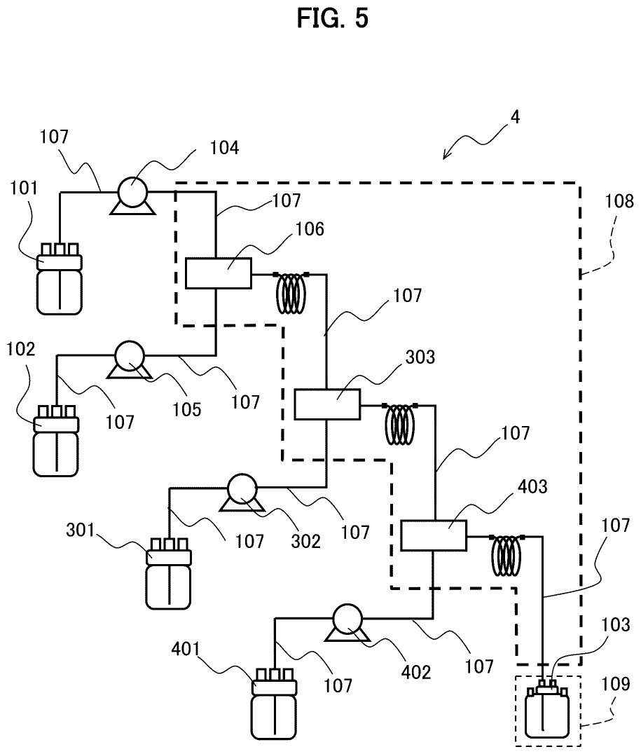

FIG. 5 is a schematic diagram of a flavan oligomer production system according to the fourth embodiment.

As shown in FIG. 5, the flavan oligomer production system 4 according to the fourth embodiment includes a raw material liquid container (first container) 101, a catalyst liquid container (second container) 102, a recovery container 103, a raw material liquid container (third container) 301, a quenching solution container (fourth container) 401, a first pump 104, a second pump 105, a third pump 302, a fourth pump 402, a first microreactor 106, a second microreactor 303, a third microreactor 403, a tube 107, a temperature controller 108, a temperature controller 109, and a not-shown fitting for connecting the tube 107 and each component.

The production system 4 is different from the production system 1 in that it includes multiple stages of microreactors 106, 303, and 403 connected in series with each other, the flavan oligomer synthesis reaction is performed stepwise, and the flavan oligomer synthesis reaction is terminated by mixing a base in the microreactor.

In the production system 4, the second microreactor 303 is connected downstream of the first microreactor 106. The third microreactor 403 is connected downstream of the second microreactor 303. The raw material liquid container 301 and the third pump 302 are connected to the second microreactor 303. The quenching solution container 401 and the fourth pump 402 are connected to the third microreactor 403.

As shown by dashed lines in the figure, the temperature controller 108 and the temperature controller 109 are provided so as to adjust a predetermined region in the system to a predetermined temperature. The first microreactor 106, the tube 107 from the first microreactor 106 to the second microreactor 303, the second microreactor 303, the tube 107 from the second microreactor 303 to the third microreactor 403, the third microreactor 403, and the tube 107 from the third microreactor 403 to the recovery container 103 are included in the adjustment range of the temperature controller 108. The recovery container 103 and the tube 107 in the recovery container 103 are included in the adjustment range of the temperature controller 109.

The first microreactor 106, the second microreactor 303, and the third microreactor 403 are flow-type reactors and have two inlets through which individual fluids are introduced from the outside, a microchannel for merging the introduced fluids, and an outlet for discharging the product fluid produced by the merging to the outside. These microreactors 106, 303, and 403 mix the fluid introduced from one inlet and the fluid introduced from the other inlet in the microchannel. By mixing the fluids, a product fluid is produced in which a predetermined reaction has begun.

In the production system 4, the raw material liquid container 101 and the first pump 104 are connected to one inlet of the first microreactor 106 via a tube 107, as in the production system 1. The catalyst liquid container 102 and the second pump 105 are connected to the other inlet of the first microreactor 106 via a tube 107, as in the production system 1.

One inlet of the second microreactor 303 is connected to the outlet of the first microreactor 106 via a tube 107. A first product fluid produced in the first microreactor 106 and the subsequent tube 107 is fed to the second microreactor 303.

The raw material liquid container 301 and the third pump 302 are connected to the other inlet of the second microreactor 303 via a tube 107. The raw material liquid container 301 is connected to the suction side of the third pump 302. The discharge side of the third pump 302 is connected to the other inlet of the second microreactor 303. The raw material liquid container 301 is provided with a raw material liquid containing a flavan derivative as a starting material. The third pump 302 feeds the raw material liquid from the raw material liquid container 301 to the other inlet of the second microreactor 303.

One inlet of the third microreactor 403 is connected to the outlet of the second microreactor 303 via a tube 107. A second product fluid produced in the second microreactor 303 and the subsequent tube 107 is fed to the third microreactor 403.

The quenching solution container 401 and the fourth pump 402 are connected to the other inlet of the third microreactor 403 via a tube 107. The quenching solution container 401 is connected to the suction side of the fourth pump 402. The discharge side of the fourth pump 402 is connected to the other inlet of the third microreactor 403. The quenching solution container 401 is provided with a quenching solution containing a base. The fourth pump 402 feeds the quenching solution from the quenching solution container 401 to the other inlet of the third microreactor 403.

The recovery container 103 is connected to the outlet of the third microreactor 403 via a tube 107. The recovery container 103 is a container for recovering a third product fluid produced in the third microreactor 403 and the subsequent tube 107. A quenching solution containing a base may or may not be stored in the recovery container 103 in order to neutralize the Lewis acid contained in the third product fluid.

As the first pump 104 and the second pump 105, an appropriate pump can be used as in the production system 1. As the third pump 302 and the fourth pump 402, an appropriate pump can be used as in the first pump 104 and the second pump 105. When a syringe pump is used as the first pump 104, the second pump 105, the third pump 302, or the fourth pump 402, a syringe provided with a raw material liquid, a catalyst liquid, a raw material liquid, or a quenching solution can be used as a functional alternative to the raw material liquid container 101, the catalyst liquid container 102, the raw material liquid container 301, or the quenching solution container 401, respectively.

As with the production system 1, appropriate materials can be used for the material of the first microreactor 106, the material of the second microreactor 303, the material of the third microreactor 403, the material of the raw material liquid container 101, the material of the catalyst liquid container 102, the material of the recovery container 103, the material of the raw material liquid container 301, the material of the quenching solution container 401, the material of the tube 107, the material of the tube, syringe, diaphragm, etc. constituting the liquid contact part of the pump, and the material of the fitting.

The microreactor 200 (see FIG. 2) capable of mixing fluids at different flow rates can be used as the first microreactor 106, the second microreactor 303, and the third microreactor 403. The microreactor 200 capable of mixing fluids at different flow rates may be used for at least one, two, or preferably all of the first microreactor 106, the second microreactor 303, and the third microreactor 403.

Next, a method for producing flavan oligomers using the flavan oligomer production system 4 will be described.

In the flavan oligomer production system 4, the raw material liquid and the catalyst liquid are mixed in the first microreactor 106 to initiate activation of the flavan derivative contained in the raw material liquid by the Lewis acid contained in the catalyst liquid. By the first mixing, a first product fluid containing the flavan derivative activated by the Lewis acid is produced. Then, the first product fluid and the raw material liquid are mixed in the second microreactor 303 to initiate a reaction between the flavan derivative activated by the Lewis acid and the unactivated flavan derivative acting as a nucleophile. By the second mixing, a second product fluid is produced in which a reaction has begun between the flavan derivatives. Then, the second product fluid and the quenching solution are mixed in the third microreactor 403 to initiate a neutralization reaction between the Lewis acid and the base. By the third mixing, a second product fluid is produced in which termination of the reaction between the flavan derivatives has begun. The flavan derivatives can be regioselectively condensed with each other by an SN1-type reaction between the activated flavan derivative and the unactivated flavan derivative, which is the nucleophile. Thereafter, the unintended polymerization reaction can be forcibly terminated by the reaction between the Lewis acid and the base.

When producing a flavan oligomer using the production system 4, a raw material liquid containing a flavan derivative having a flavan skeleton is prepared in the raw material liquid container 101. A catalyst liquid containing a Lewis acid is prepared in the catalyst liquid container 102. A raw material liquid containing a flavan derivative having a flavan skeleton is prepared in the raw material liquid container 301. A quenching solution containing a base is prepared in the quenching solution container 401. A quenching solution containing a base may or may not be prepared in the recovery container 103.

First, the raw material liquid containing the flavan derivative prepared in the raw material liquid container 101 is fed from the raw material liquid container 101 to one inlet of the first microreactor 106 by the first pump 104. Further, the catalyst liquid containing the Lewis acid prepared in the catalyst liquid container 102 is fed from the catalyst liquid container 102 to the other inlet of the first microreactor 106 by the second pump 105.

Next, the raw material liquid containing the flavan derivative and the catalyst liquid containing the Lewis acid are mixed in the first microreactor 106. By the mixing, activation of the flavan derivative contained in the raw material liquid by the Lewis acid contained in the catalyst liquid is initiated. The activation of the flavan derivative further proceeds while the first product fluid produced in the first microreactor 106 flows in the subsequent tube 107 toward the downstream. The first product fluid is fed from the first microreactor 106 to one inlet of the second microreactor 303.

In the first microreactor 106 of the production system 4, the flavan derivative, which is a starting material, and the Lewis acid are reacted at a reaction equivalent ratio close to flavan derivative:Lewis acid=1:1. Therefore, the flow rate ratio of the raw material liquid and the catalyst liquid introduced into the first microreactor 106, the concentration of the raw material liquid prepared in the raw material liquid container 101, and the concentration of the catalyst liquid prepared in the catalyst liquid container 102 are adjusted so as to achieve such a reaction equivalent ratio. The reaction equivalent ratio of the flavan derivative and the Lewis acid may be adjusted only by the flow rate ratio, may be adjusted only by the concentration, or may be adjusted by both the flow rate ratio and the concentration.

Subsequently, the raw material liquid containing the flavan derivative prepared in the raw material liquid container 301 is fed from the raw material liquid container 301 to the other inlet of the second microreactor 303 by the third pump 302.

Next, the first product fluid containing the activated flavan derivative and the raw material liquid containing the unactivated flavan derivative acting as a nucleophile are mixed in the second microreactor 303. By the mixing, a reaction between the activated flavan derivative and the unactivated flavan derivative acting as a nucleophile is initiated. The reaction between the flavan derivatives further proceeds while the second product fluid produced in the second microreactor 303 flows through the subsequent tube 107 toward the downstream. The second product fluid is fed from the second microreactor 303 to one inlet of the third microreactor 403.

In the second microreactor 303 of the production system 4, the flavan derivative activated in the first microreactor 106 and the subsequent tube 107 and the unactivated flavan derivative prepared in the raw material liquid container 301 are reacted at a reaction equivalent ratio close to activated form:unactivated form=1:1. Therefore, the flow rate ratio of the first product fluid and the raw material liquid introduced into the second microreactor 303 and the concentration of the raw material liquid prepared in the raw material liquid container 301 are adjusted so as to achieve such a reaction equivalent ratio. The reaction equivalent ratio of the activated flavan derivative and the unactivated flavan derivative may be adjusted only by the flow rate ratio, may be adjusted only by the concentration, or may be adjusted by both the flow rate ratio and the concentration.

Subsequently, the quenching solution containing the base prepared in the quenching solution container 401 is fed from the quenching solution container 401 to the other inlet of the third microreactor 403 by the fourth pump 402.

Next, the second product fluid in which the polymerization reaction is in progress and the quenching solution containing the base are mixed in the third microreactor 403. By the mixing, a neutralization reaction between the Lewis acid and the base is initiated. The neutralization reaction further proceeds while the third product fluid produced in the third microreactor 403 flows in the subsequent tube 107 toward the downstream. By the neutralization, the reaction between the unactivated flavan derivative and the Lewis acid and the reaction between the oligomer of the flavan derivative produced in the polymerization reaction and the Lewis acid are terminated.

In the third microreactor 403 of the production system 4, the Lewis acid in the second product fluid produced in the second microreactor 303 and the subsequent tube 107 and the base prepared in the quenching solution container 401 are reacted so that the base is 1 equivalent or more with respect to 1 equivalent of the Lewis acid. Therefore, the flow rate ratio of the second product fluid and the quenching solution introduced into the third microreactor 403 and the concentration of the quenching solution prepared in the quenching solution container 401 are adjusted so as to achieve such a reaction equivalent ratio. The reaction equivalent ratio of the Lewis acid and the base may be adjusted only by the flow rate ratio, may be adjusted only by the concentration, or may be adjusted by both the flow rate ratio and the concentration.

Subsequently, the third product fluid discharged from the third microreactor 403 and the subsequent tube 107 is recovered into the recovery container 103. When the polymerization reaction is initiated in the microchannel of the second microreactor 303, the reaction proceeds through the subsequent tube 107, the polymerization reaction termination is initiated in the microchannel of the third microreactor 403, and the reaction proceeds through the subsequent tube 107, an oligomer is obtained in which the flavan derivatives are bonded at a desired degree of polymerization.

The produced oligomer of the flavan derivative can be deprotected by removing the protecting group after separation and purification, if necessary. The oligomer of the flavan derivative can be subjected to a step of introducing a new substituent, a step of introducing a modified structure such as a sugar chain, other reaction steps of converting a three-dimensional structure, a skeleton, a functional group, or the like, and the like before or after the deprotection.

According to the flavan oligomer production system 4 and the flavan oligomer production method described above, the flavan derivative contained in the raw material liquid and the Lewis acid contained in the catalyst liquid can efficiently react in the microchannel of the first microreactor 106 and the subsequent tube 107, and a first product fluid is produced. Further, the activated flavan derivative contained in the first product fluid and the flavan derivative contained in the raw material liquid can efficiently react in the microchannel of the second microreactor 303 and the subsequent tube 107, and a second product fluid is produced. Then, the Lewis acid contained in the second product fluid and the base contained in the quenching solution can efficiently react in the third microreactor 403 and the subsequent tube 107. Therefore, the flavan derivative and the Lewis acid can be reacted at a predetermined reaction equivalent ratio, the produced activated flavan derivative and the flavan derivative acting as a nucleophile can be reacted at a predetermined reaction equivalent ratio, and then the Lewis acid can be neutralized to terminate the multimerization reaction of the flavan derivative. By using a microreactor, it is possible to precisely control the flow rate ratio of the fluids, the reaction ratio of the reactants, the initiation time of the reaction, and the termination time of the reaction. Therefore, a flavan oligomer in which flavan derivatives are bonded at a desired degree of polymerization can be efficiently synthesized in high yield.

Further, according to the flavan oligomer production system 4 and the flavan oligomer production method described above, since the yield of the oligomer having the desired degree of polymerization is improved, it is possible to reduce the cost and labor for separating and purifying the oligomer having the desired degree of polymerization from the mixture after the synthesis. Further, an excess amount of catalyst is not required, and the cost of the catalyst and the purification cost are reduced. According to the flavan oligomer production system 4 and the flavan oligomer production method, control of the reaction conditions becomes easier and the reaction can be terminated rapidly and reliably compared to the production system 1.

The reaction equivalent ratio of the flavan derivative and the Lewis acid to be reacted in the first microreactor 106 of the production system 4 and the tube 107 on the downstream side of the first microreactor 106 can be set to the same conditions as in the production system 2. The reaction equivalent ratio of the flavan derivative and the Lewis acid depends on the types of the flavan derivative and the Lewis acid, but from the viewpoint of saving the amount of the Lewis acid used, the ratio is preferably flavan derivative:Lewis acid=1:1 to 1:2.

The reaction equivalent ratio of the activated flavan derivative and the flavan derivative acting as a nucleophile to be reacted in the second microreactor 303 of the production system 4 and the tube 107 on the downstream side of the second microreactor 303 can be set to the same conditions as in the production system 2. The reaction equivalent ratio of the activated flavan derivative and the flavan derivative acting as a nucleophile depends on the types of the flavan derivative and the Lewis acid, but from the viewpoint of saving the amount of the flavan derivative used, the ratio is preferably activated flavan derivative:flavan derivative acting as a nucleophile=1:1 to 1:2.

The reaction equivalent ratio of the Lewis acid and the base to be reacted in the third microreactor 403 of the production system 4 and the tube 107 on the downstream side of the third microreactor 403 can be set to the same conditions as in the production system 3. The reaction equivalent ratio of the base is preferably 1 equivalent or more, more preferably an excess amount exceeding 1 equivalent, with respect to 1 equivalent of the Lewis acid to be reacted in the first microreactor 106.

### Fifth Embodiment

Next, a flavan oligomer production system and a flavan oligomer production method according to the fifth embodiment of the present invention will be described with reference to the drawings.

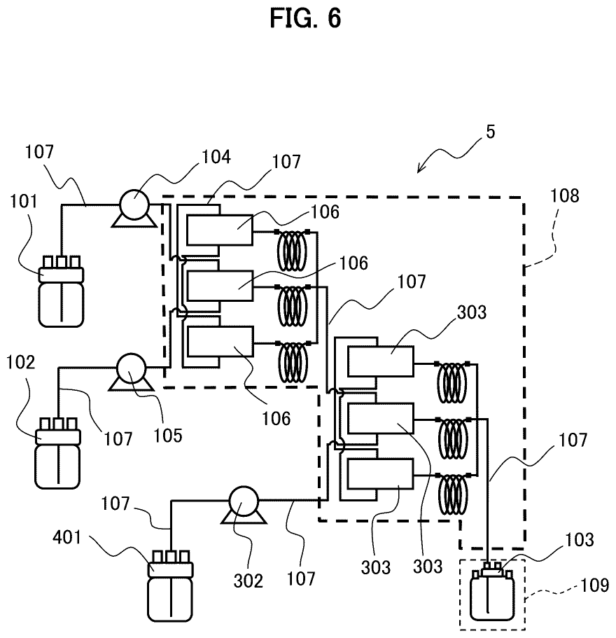

FIG. 6 is a schematic diagram of a flavan oligomer production system according to the fifth embodiment.

As shown in FIG. 6, the flavan oligomer production system 5 according to the fifth embodiment includes a raw material liquid container (first container) 101, a catalyst liquid container (second container) 102, a recovery container 103, a quenching solution container (fourth container) 401, a first pump 104, a second pump 105, a third pump 302, a first microreactor 106, a second microreactor 303, a tube 107, a temperature controller 108, a temperature controller 109, and a not-shown fitting for connecting the tube 107 and each component, as in the production system 3.

The production system 5 is different from the production system 3 in that it includes multiple stages of microreactors 106 and 303 connected in series with each other, and multiple microreactors 106 and 303 are arranged in parallel at each stage.

In the production system 5, three microreactors are arranged in parallel as the first microreactor 106. Three microreactors are arranged in parallel as the second microreactor 303.

In the production system 5, a first branch header is connected to one inlet of each of the first microreactors 106 arranged in parallel. The raw material liquid container 101 and the first pump 104 are connected to the first branch header via a tube 107, as in the production system 3. A second branch header is connected to the other inlet of each of the first microreactors 106 arranged in parallel. The catalyst liquid container 102 and the second pump 105 are connected to the second branch header via a tube 107, as in the production system 3.

A first merging header is connected to the outlet of each of the first microreactors 106 arranged in parallel. A third branch header is connected to the first merging header via a tube 107. A first product fluid produced in the first microreactor 106 and the subsequent tube 107 merges at the first merging header and is fed to the third branch header.

The third branch header is connected to one inlet of each of the second microreactors 303 arranged in parallel. A fourth branch header is connected to the other inlet of each of the second microreactors 303 arranged in parallel. The quenching solution container 401 and the third pump 302 are connected to the fourth branch header via a tube 107.

A second merging header is connected to the outlet of each of the second microreactors 303 arranged in parallel. The recovery container 103 is connected to the second merging header via a tube 107. The recovery container 103 is a container for recovering a second product fluid produced in the second microreactor 303 and the subsequent tube 107. A quenching solution containing a base may or may not be stored in the recovery container 103 in order to neutralize the Lewis acid contained in the second product fluid.

In the flavan oligomer production system 5, mixing of the raw material liquid and the catalyst liquid is initiated in each of the first microreactors 106 arranged in parallel, and a first product fluid is produced. Then, mixing of the first product fluid and the quenching solution is initiated in each of the second microreactors 303 arranged in parallel, and a second product fluid is produced.

The flow rate of each fluid introduced into the first microreactors 106 arranged in parallel is preferably controlled to be the same flow rate. Further, the flow rate of each fluid introduced into the second microreactors 303 arranged in parallel is preferably controlled to be the same flow rate. With such control, the reaction time can be accurately and stably managed for the microreactors arranged in parallel.

The timing of mixing in the first microreactors 106 arranged in parallel is preferably controlled to be the same. Further, the timing of mixing in the second microreactors 303 arranged in parallel is preferably controlled to be the same. With such control, the reaction time can be appropriately aligned for the microreactors arranged in parallel.

According to the flavan oligomer production system 5 and the flavan oligomer production method described above, since multiple microreactors 106 and 303 are arranged in parallel at each stage, it is possible to increase the overall fluid throughput while utilizing the micro reaction field for mixing the fluids. Since a large volume of fluid can be mixed as a whole, a flavan derivative oligomer having a desired degree of polymerization can be synthesized in high yield in a large amount regardless of the concentrations of the raw material liquid, the catalyst liquid, the quenching solution, and the like.

Although the production system 5 includes microreactors 106 and 303 arranged in parallel in three, the number of microreactors arranged in parallel can be an appropriate number of 2 or more. By numbering up by arranging the microreactors in parallel, the overall production amount can be easily increased while maintaining the yield of the oligomer having the predetermined degree of polymerization.

The number of microreactors arranged in parallel may be the same or different for each stage. The number of microreactors arranged in parallel can be changed to an appropriate number according to the type of microreactor, the target production amount of the oligomer having the predetermined degree of polymerization, and the like.

When the number of microreactors arranged in parallel is the same for each stage, the fluid produced in the microreactor of the previous stage can be fed to the microreactor of the next stage without merging or dividing. When the number of microreactors arranged in parallel is the same for each stage or different from each other, the fluid produced in the microreactor of the previous stage can be partially merged or divided and then fed to the microreactor of the next stage. The merging header or the branch header used at the time of merging or dividing may be a combination of multiple merging headers or a combination of multiple branch headers.

Further, although the production system 5 arranges the microreactors 106 and 303 of the production system 3 in parallel, the microreactor 106 of the production system 1, the microreactors 106 and 303 of the production system 2, or the microreactors 106, 303, and 403 of the production system 4 may be arranged in parallel.

Although the embodiments of the present invention have been described above, the present invention is not limited to the above embodiments, and various modifications can be made without departing from the spirit of the present invention. For example, the present invention is not necessarily limited to one having all the configurations included in the above embodiments. A part of the configuration of a certain embodiment may be replaced with another configuration, a part of the configuration of a certain embodiment may be added to another embodiment, or a part of the configuration of a certain embodiment may be omitted.

For example, the flavan oligomer production system described above may have a configuration in which four or more microreactors are connected in series. Any of the configurations of the production systems 1 to 5 can be repeatedly connected in series for each type of flavan derivative to be polymerized. A k-th microreactor (k is an integer of 2 or larger than 2) for mixing a mixed fluid produced in the microreactor of the previous stage and a fluid to be mixed prepared in a container is connected in series to the downstream side of the first microreactor, and a recovery container can be provided at the final stage. According to such a configuration, a 2m-mer (m is an integer of 1 or larger than 1) in which flavan derivatives are bonded with a desired degree of polymerization can be synthesized.

Further, in the case of a configuration in which the flavan oligomer production system described above performs a stepwise synthesis reaction (see FIG. 3 and the like), flavan derivatives having different degrees of polymerization may be prepared in each raw material liquid container and polymerized with each other. For example, a combination of all activated flavan derivative monomers and unactivated flavan derivative dimers, a combination of all activated flavan derivative dimers and unactivated flavan derivative monomers, and the like can be reacted. According to such a configuration, a 2m+1-mer (m is an integer of 1 or larger than 1) in which flavan derivatives are bonded with a desired degree of polymerization can be synthesized.

Further, the flavan oligomer production system described above may include a fluid detection sensor for detecting the arrival of fluid at the microreactor. By using the fluid detection sensor, the arrival of the fluid at the microreactor through the tube can be detected, so that the fluids can be introduced to the merging point of the microreactor at the same time. By performing such control, the prepared fluids can be mixed without excess or deficiency, so that the entire amount of the prepared fluids can be reacted at a predetermined reaction equivalent ratio. It is also possible to omit the disposal of the unreacted portion that did not react at the desired reaction equivalent ratio.

### EXAMPLES

Hereinafter, the present invention will be specifically described with reference to Examples, but the technical scope of the present invention is not limited thereto.

The production of the oligomer of the flavan derivative was carried out using the production system 3 (see FIG. 4). Further, for comparison, the production of the oligomer of the flavan derivative by a batch method was carried out.

### Example 1

Example 1 was carried out using a microreactor in the configuration of the production system 3 (see FIG. 4). As the first microreactor 106, the microreactor 200 (see FIG. 2) capable of mixing fluids at different flow rates was used. The microreactor 200 was made of quartz glass (manufactured by Hitachi Plant Services Co., Ltd.). The flow path width and flow path depth of the mixed flow path 205 of the microreactor 200 were set to 0.2 mm.

As the second microreactor 303, a PEEK T-shaped reactor YMC-P-0021 (manufactured by YMC Co., Ltd.) was used.

As the first pump 104, the second pump 105, and the third pump 302, a syringe pump Model 11 Single Syringe Pump 55-1199 (manufactured by Harvard Apparatus) and a syringe Model 11 Single Syringe 70-2208 (manufactured by Harvard Apparatus) were used.

As the tube 107, a PTFE tube (manufactured by GL Sciences Inc.) having an outer diameter of 1/16 inch and an inner diameter of 0.5 mm was used. The length of the tube 107 from the first microreactor 106 to the second microreactor 303 was set to 0.3 m or 0.9 m. The length of the tube 107 from the second microreactor 303 to the recovery container 103 was set to 0.2 m.

The temperature controller 108 was adjusted to −70° C. using a mixed bath using dry ice and a water/methanol mixed solvent. The temperature controller 109 was adjusted to 0° C. using an ice bath.

As the monomer of the flavan derivative as a starting material, a compound represented by the following general formula (5) was used. In general formula (5), Bn* represents a benzyl group labeled with deuterium.

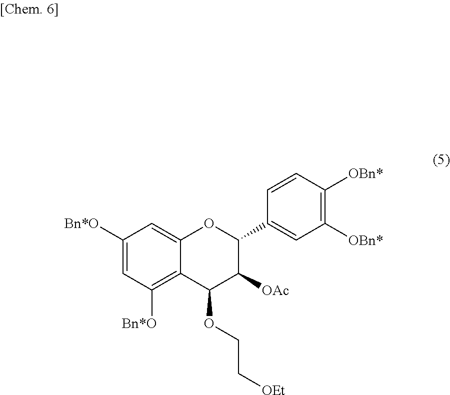

Trimethylsilyl trifluoromethanesulfonate (TMSOTf) was used as the Lewis acid. Triethylamine (Et₃N) was used as the base. Dichloromethane was used as the solvent.

A raw material liquid containing 0.05 M of the monomer of the flavan derivative was prepared in the raw material liquid container 101. A catalyst liquid containing 0.05 M of the Lewis acid was prepared in the catalyst liquid container 102. A quenching solution containing 0.3 M of the base was prepared in the quenching solution container 401.

The raw material liquid containing the monomer of the flavan derivative and the catalyst liquid containing the Lewis acid were mixed in the first microreactor 106, and then the first product fluid in which the flavan derivatives were reacting and the quenching solution containing the base were mixed in the second microreactor 303. In the first microreactor 106, the raw material liquid containing the monomer of the flavan derivative was introduced from the high flow rate side fluid inlet 207, and the catalyst liquid containing the Lewis acid was introduced from the low flow rate side fluid inlet 208. The flow rate of the raw material liquid was set to 1 mL/min. The flow rate of the catalyst liquid was set to 1 mL/min. The flow rate of the quenching solution was set to 1 mL/min. Therefore, the equivalent ratio of the monomer of the flavan derivative and the Lewis acid was 1:1, and the equivalent ratio of the introduced Lewis acid and the base was 1:6.

The reaction time of the flavan derivative was adjusted by the length of the tube 107. When the length of the tube 107 from the first microreactor 106 to the second microreactor 303 is 0.3 m, it corresponds to a reaction time of 1.77 s. When the length of the tube 107 from the first microreactor 106 to the second microreactor 303 is 0.9 m, it corresponds to a reaction time of 5.30 s.

A quenching solution in which 1 mL of triethylamine (Et₃N) was dissolved in 2 mL of dichloromethane was placed in the recovery container 103 in order to completely terminate the reaction.

### Comparative Example 1

Comparative Example 1 was carried out by a batch method. A monomer of the flavan derivative was placed in a two-necked flask having a volume of 5 mL. The inside of the flask was depressurized and purged with argon gas, and then capped with a septum cap. Dichloromethane was added into the flask by piercing the septum with a syringe to prepare 0.4 mL of a raw material liquid containing 0.05 M of the monomer of the flavan derivative. The two-necked flask was immersed in a mixed bath using dry ice and a water/methanol mixed solvent at −70° C.

Then, while stirring at 400 to 500 rpm, 0.4 mL of a catalyst liquid containing 0.05 M of the Lewis acid was added with a syringe along the inner wall. The reaction time was set to 5 s or 5 min. After the lapse of a predetermined reaction time, 0.3 mL of a quenching solution containing a base was added to terminate the reaction.

As the monomer of the flavan derivative as a starting material, a compound represented by general formula (5) was used. Trimethylsilyl trifluoromethanesulfonate (TMSOTf) was used as the Lewis acid. Triethylamine (Et₃N) was used as the base. Dichloromethane was used as the solvent. The equivalent ratio of the monomer of the flavan derivative and the Lewis acid was 1:1.

### Experimental Results

In Example 1 and Comparative Example 1, a mixture containing an oligomer of the flavan derivative was obtained after the reaction of the monomer of the flavan derivative. The mixture contained a dimer of the flavan derivative, higher-order oligomers such as a trimer or higher, an unreacted monomer, and the like. The dimer of the flavan derivative is represented by the following general formula (6). In general formula (6), Bn* represents a benzyl group labeled with deuterium.

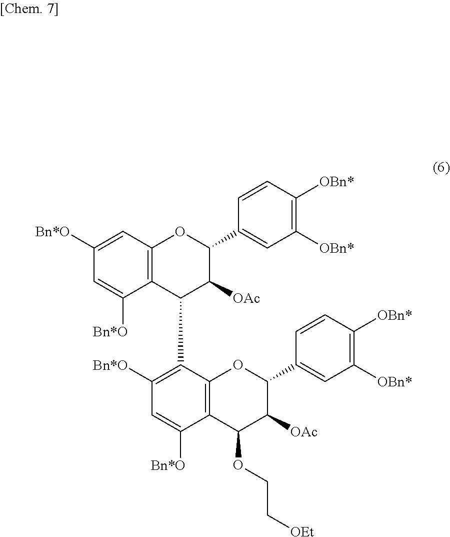

Table 1 shows the reaction time of the synthesis reaction, the yield of the dimer of the flavan derivative, the yield of the trimer of the flavan derivative, the weight yield of higher-order oligomers of the flavan derivative such as a tetramer or higher, and the proportion of the unreacted monomer of the flavan derivative. The yield is the ratio of the actual yield to the maximum yield (theoretical yield) produced from the starting material, which is 100%.

TABLE 1

| | | | | | Production | Proportion |
|---|---|---|---|---|---|---|
| | | | | | Amount of | of |
| | | | Dime | Trimer | Higher-Order | Unreacted |
| | Reaction | | Yield | Yield | Oligomer | Monomer |
| | Time | | [%] | [%] | [wt %] | [%] |
| Microreactor | 1.77 | s | 43 | — | 10 | 30 |
| (Production | 5.30 | s | 62 | — | 5 | 19 |
| System) | | | | | | |
| Batch | 5 | s | 56 | — | 4 | 5 |
| Method | 5 | min | 20 | 10 | 29 | — |

As shown in Table 1, when a microreactor was used, the yield of the dimer was 43% at a reaction time of 1.77 s, no trimer was produced, the weight yield of higher-order oligomers such as a tetramer or higher was 10 wt %, and the proportion of the unreacted monomer was 30%. At a reaction time of 5.30 s, the yield of the dimer improved to 62%, no trimer was produced, the weight yield of higher-order oligomers such as a tetramer or higher was reduced to 5 wt %, and the proportion of the unreacted monomer was reduced to 19%. In any reaction time, the production of higher-order oligomers such as a trimer or higher was suppressed.

On the other hand, when the batch method was used, the yield of the dimer was 56% at a reaction time of 5 s, no trimer was produced, the weight yield of higher-order oligomers such as a tetramer or higher was 4 wt %, and the proportion of the unreacted monomer was 5%. At a reaction time of 5 min, the yield of the dimer was 20%, the yield of the trimer was 10%, and the weight yield of higher-order oligomers such as a tetramer or higher was 29 wt %. As the reaction time elapsed, trimers and higher-order oligomers such as a tetramer or higher were rapidly produced, making it difficult to control the degree of polymerization.

When using the batch method, it may be possible to precisely control the reaction time when the reaction vessel is small. However, in order to increase the production amount, it is necessary to increase the size of the reaction vessel. As the size of the reaction vessel increases, it becomes difficult to uniformly mix and control the reaction time, resulting in variations in the polymerization reaction. If the reaction time is short, the reaction does not proceed, and if the reaction time is long, the yield of the dimer (oligomer having the predetermined degree of polymerization) decreases. Therefore, it can be said that when the reaction vessel is enlarged to increase the production amount, the yield of the dimer (oligomer having the predetermined degree of polymerization) per the same reaction time decreases.

On the other hand, when using a microreactor, it can be said that the yield of the dimer (oligomer having the predetermined degree of polymerization) improves because the reaction time can be precisely controlled. When using a microreactor, it can be said that the production amount can be increased while maintaining the yield because numbering up by parallelization is easy.

### REFERENCE SIGNS LIST

1, 2, 3, 4, 5 flavan oligomer production system
101 raw material liquid container (first container)
102 catalyst liquid container (second container)
103 recovery container
104 first pump
105 second pump
106 (first) microreactor
107 tube
108 temperature controller
109 temperature controller
200 microreactor
201 upper plate
202 lower plate
203 high flow rate side flow path (microchannel)
204 low flow rate side flow path (microchannel)
205 mixed flow path (microchannel)
206 merging point
207 high flow rate side fluid inlet (through hole)
208 low flow rate side fluid inlet (through hole)
209 fluid outlet (through hole)
301 raw material liquid container (third container)
302 third pump
303 (second) microreactor
401 quenching solution container (fourth container)
402 fourth pump
403 (third) microreactor

1. A flavan oligomer production system for producing a flavan oligomer containing flavan derivatives which include a flavan skeleton and which are bonded to each other, the flavan oligomer production system comprising:
at least one microreactor including two inlets allowing fluids to be introduced into the two inlets and a flow path allowing the fluids to merge in the flow path, the at least one microreactor being configured to mix in the flow path a first fluid to be introduced from one of the inlets and a second fluid to be introduced from another of the inlets;
a first container in which the first fluid is to be disposed;
a second container in which the second fluid is to be disposed; and
a recovery container configured to recover a product fluid to be produced in the at least one microreactor,
wherein the first fluid is a liquid containing a flavan derivative including a flavan skeleton,
wherein the second fluid is a liquid containing a Lewis acid, and
wherein the product fluid includes an oligomer containing the flavan derivatives bonded to each other and is to be recovered in a liquid in the recovery container, the liquid containing a base.

2. A flavan oligomer production system for producing a flavan oligomer containing flavan derivatives which include a flavan skeleton and which are bonded to each other, the flavan oligomer production system comprising:
at least one first microreactor including two inlets allowing fluids to be introduced into the two inlets and a flow path allowing the fluids to merge in the flow path, the at least one first microreactor being configured to mix in the flow path a first fluid to be introduced from one of the inlets and a second fluid to be introduced from another of the inlets;
at least one second microreactor including two inlets allowing fluids to be introduced into the two inlets and a flow path allowing the fluids to merge in the flow path, the at least one second microreactor being configured to mix in the flow path a third fluid to be introduced from one of the inlets and a first product fluid to be produced in the at least one first microreactor and to be introduced from another of the inlets;
a first container in which the first fluid is to be disposed;
a second container in which the second fluid is to be disposed;
a third container in which the third fluid is to be disposed; and
a recovery container configured to recover a second product fluid to be produced in the at least one second microreactor,
wherein the first fluid is a liquid containing a flavan derivative including a flavan skeleton,
wherein the second fluid is a liquid containing a Lewis acid,
wherein the third fluid is a liquid containing a flavan derivative including a flavan skeleton, and
wherein the second product fluid includes an oligomer containing the flavan derivatives bonded to each other and is to be recovered in a liquid in the recovery container, the liquid containing a base.

3. A flavan oligomer production system for producing a flavan oligomer containing flavan derivatives which include a flavan skeleton and which are bonded to each other, the flavan oligomer production system comprising:
at least one first microreactor including two inlets allowing fluids to be introduced into the two inlets and a flow path allowing the fluids to merge in the flow path, the at least one first microreactor being configured to mix in the flow path a first fluid to be introduced from one of the inlets and a second fluid to be introduced from another of the inlets;
at least one second microreactor including two inlets allowing fluids to be introduced into the two inlets and a flow path allowing the fluids to merge in the flow path, the at least one second microreactor being configured to mix in the flow path a third fluid to be introduced from one of the inlets and a first product fluid to be produced in the at least one first microreactor and to be introduced from another of the inlets;
a first container in which the first fluid is to be disposed;
a second container in which the second fluid is to be disposed;
a third container in which the third fluid is to be disposed; and
a recovery container configured to recover a second product fluid to be produced in the at least one second microreactor,
wherein the first fluid is a liquid containing a flavan derivative including a flavan skeleton,
wherein the second fluid is a liquid containing a Lewis acid,
wherein the third fluid is a liquid containing a base, and
wherein the second product fluid includes an oligomer containing the flavan derivatives bonded to each other and is to be recovered in the recovery container.

4. A flavan oligomer production system for producing a flavan oligomer containing flavan derivatives which include a flavan skeleton and which are bonded to each other, the flavan oligomer production system comprising:
at least one first microreactor including two inlets allowing fluids to be introduced into the two inlets and a flow path allowing the fluids to merge in the flow path, the at least one first microreactor being configured to mix in the flow path a first fluid to be introduced from one of the inlets and a second fluid to be introduced from another of the inlets;
at least one second microreactor including two inlets allowing fluids to be introduced into the two inlets and a flow path allowing the fluids to merge in the flow path, the at least one second microreactor being configured to mix in the flow path a third fluid to be introduced from one of the inlets and a first product fluid to be produced in the at least one first microreactor and to be introduced from another of the inlets;
at least one third microreactor including two inlets allowing fluids to be introduced into the two inlets and a flow path allowing the fluids to merge in the flow path, the at least one third microreactor being configured to mix in the flow path a fourth fluid to be introduced from one of the inlets and a second product fluid to be produced in the at least one second microreactor and to be introduced from another of the inlets;
a first container in which the first fluid is to be disposed;
a second container in which the second fluid is to be disposed;
a third container in which the third fluid is to be disposed;
a fourth container in which the fourth fluid is to be disposed; and
a recovery container configured to recover a third product fluid to be produced in the at least one third microreactor,
wherein the first fluid is a liquid containing a flavan derivative including a flavan skeleton,
wherein the second fluid is a liquid containing a Lewis acid,
wherein the third fluid is a liquid containing a flavan derivative including a flavan skeleton,
wherein the fourth fluid is a liquid containing a base, and
wherein the third product fluid includes an oligomer containing the flavan derivatives bonded to each other and is to be recovered in the recovery container.

5. The flavan oligomer production system according to claim 1, wherein the flavan derivative is a monomer including one flavan skeleton or an oligomer including two or more flavan skeletons.

6. The flavan oligomer production system according to claim 1, wherein the flavan derivative is a compound represented by the following general formula (1) or (2): 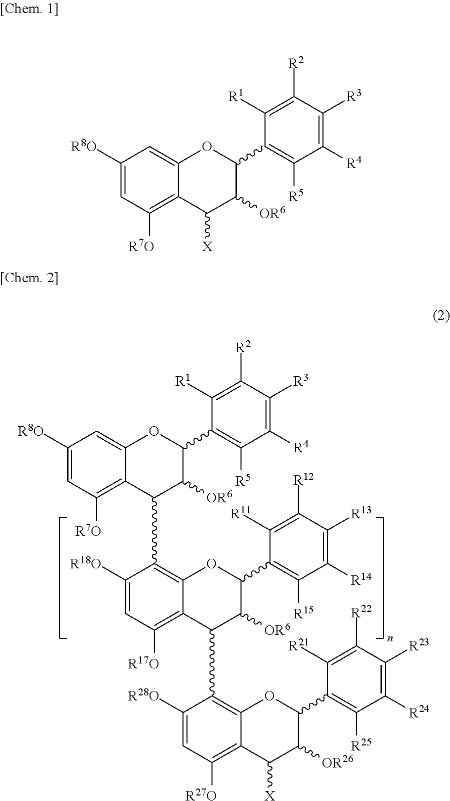
wherein in the general formula (1), R¹ to R⁵ each independently represent a hydrogen atom, a hydroxy group, an alkoxy group, or a substituent represented by OR⁹; R⁶ represents a hydrogen atom, a hydrocarbon group which may include a substituent, an alkoxyalkyl group, an acyl group, a silyl group, or a galloyl group; R⁷ to R⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration, and
wherein in the general formula (2), R¹ to R⁵, R¹¹ to R¹⁵, and R²¹ to R²⁵ each independently represent a hydrogen atom, a hydroxy group, an alkoxy group, or a substituent represented by OR⁹; R⁶, R¹⁶, and R²⁶ represents a hydrogen atom, a hydrocarbon group which may include a substituent, an alkoxyalkyl group, an acyl group, a silyl group, or a galloyl group; R⁷ to R⁸, R¹⁷ to R¹⁸, and R²⁷ to R²⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration; n represents an integer of 0 or larger than 0.

7. A flavan oligomer production method for producing a flavan oligomer containing flavan derivatives which include a flavan skeleton and which are bonded to each other, the flavan oligomer production method comprising, in a microreactor system including at least one microreactor which includes two inlets allowing fluids to be introduced into the two inlets and a flow path allowing the fluids to merge in the flow path and which is configured to mix in the flow path a first fluid to be introduced from one of the inlets and a second fluid to be introduced from another of the inlets, a first container in which the first fluid is to be disposed, a second container in which the second fluid is to be disposed, and a recovery container configured to recover a product fluid to be produced in the at least one microreactor:
preparing as the first fluid a liquid containing a flavan derivative including a flavan skeleton;
preparing as the second fluid a liquid containing a Lewis acid;
mixing the first fluid and the second fluid in the microreactor;
activating a part of the flavan derivative of the first fluid by using the Lewis acid of the second fluid;
initiating a reaction between an activated part of the flavan derivative of the first fluid and a remaining part of the flavan derivative of the first fluid acting as a nucleophile;
recovering in a liquid containing a base the product fluid being reacting;
stopping a reaction of the product fluid by using the base; and
producing an oligomer containing the flavan derivatives bonded to each other.

8. A flavan oligomer production method for producing a flavan oligomer containing flavan derivatives which include a flavan skeleton and which are bonded to each other, the flavan oligomer production method comprising, in a microreactor system including at least one first microreactor which includes two inlets allowing fluids to be introduced into the two inlets and a flow path allowing the fluids to merge in the flow path and which is configured to mix in the flow path a first fluid to be introduced from one of the inlets and a second fluid to be introduced from another of the inlets, at least one second microreactor which includes two inlets allowing fluids to be introduced into the two inlets and a flow path allowing the fluids to merge in the flow path and which is configured to mix in the flow path a third fluid to be introduced from one of the inlets and a first product fluid to be produced in the at least one first microreactor and to be introduced from another of the inlets, a first container in which the first fluid is to be disposed, a second container in which the second fluid is to be disposed, a third container in which the third fluid is to be disposed, and a recovery container configured to recover a second product fluid to be produced in the at least one second microreactor:
preparing as the first fluid a liquid containing a flavan derivative including a flavan skeleton;
preparing as the second fluid a liquid containing a Lewis acid;
preparing as the third fluid a liquid containing a flavan derivative including a flavan skeleton;
mixing the first fluid and the second fluid in the at least one first microreactor;
activating the flavan derivative of the first fluid by using the Lewis acid of the second fluid;
mixing the first product fluid and the third fluid in the at least one second microreactor;
initiating a reaction between an activated flavan derivative of the first product fluid and the flavan derivative of the third fluid acting as a nucleophile;
recovering in a liquid containing a base the second product fluid being reacting;
stopping a reaction of the second product fluid by using the base; and
producing an oligomer containing the flavan derivatives bonded to each other.

9. A flavan oligomer production method for producing a flavan oligomer containing flavan derivatives which include a flavan skeleton and which are bonded to each other, the flavan oligomer production method comprising, in a microreactor system including at least one first microreactor which includes two inlets allowing fluids to be introduced into the two inlets and a flow path allowing the fluids to merge in the flow path and which is configured to mix in the flow path a first fluid to be introduced from one of the inlets and a second fluid to be introduced from another of the inlets, at least one second microreactor which includes two inlets allowing fluids to be introduced into the two inlets and a flow path allowing the fluids to merge in the flow path and which is configured to mix in the flow path a third fluid to be introduced from one of the inlets and a first product fluid to be produced in the at least one first microreactor and to be introduced from another of the inlets, a first container in which the first fluid is to be disposed, a second container in which the second fluid is to be disposed, a third container in which the third fluid is to be disposed, and a recovery container configured to recover a second product fluid to be produced in the at least one second microreactor:
preparing as the first fluid a liquid containing a flavan derivative including a flavan skeleton;
preparing as the second fluid a liquid containing a Lewis acid;
preparing as the third fluid a liquid containing a base;
mixing the first fluid and the second fluid in the at least one first microreactor;
activating a part of the flavan derivative of the first fluid by using the Lewis acid of the second fluid;
initiating a reaction between an activated part of the flavan derivative of the first fluid and a remaining part of the flavan derivative of the first fluid acting as a nucleophile;
mixing the first product fluid and the third fluid in the at least one second microreactor;
stopping a reaction of the first product fluid by using the base; and
producing an oligomer containing the flavan derivatives bonded to each other.

10. A flavan oligomer production method for producing a flavan oligomer containing flavan derivatives which include a flavan skeleton and which are bonded to each other, the flavan oligomer production method comprising, in a microreactor system including at least one first microreactor which includes two inlets allowing fluids to be introduced into the two inlets and a flow path allowing the fluids to merge in the flow path and which is configured to mix in the flow path a first fluid to be introduced from one of the inlets and a second fluid to be introduced from another of the inlets, at least one second microreactor which includes two inlets allowing fluids to be introduced into the two inlets and a flow path allowing the fluids to merge in the flow path and which is configured to mix in the flow path a third fluid to be introduced from one of the inlets and a first product fluid to be produced in the at least one first microreactor and to be introduced from another of the inlets, at least one third microreactor which includes two inlets allowing fluids to be introduced into the two inlets and a flow path allowing the fluids to merge in the flow path and which is configured to mix in the flow path a fourth fluid to be introduced from one of the inlets and a second product fluid to be produced in the at least one second microreactor and to be introduced from another of the inlets, a first container in which the first fluid is to be disposed, a second container in which the second fluid is to be disposed, a third container in which the third fluid is to be disposed, a fourth container in which the fourth fluid is to be disposed, and a recovery container configured to recover a third product fluid to be produced in the at least one third microreactor:
preparing as the first fluid a liquid containing a flavan derivative including a flavan skeleton;
preparing as the second fluid a liquid containing a Lewis acid;
preparing as the third fluid a liquid containing a flavan derivative including a flavan skeleton;
preparing as the fourth fluid a liquid containing a base;
mixing the first fluid and the second fluid in the at least one first microreactor;
activating the flavan derivative of the first fluid by using the Lewis acid of the second fluid;
mixing the first product fluid and the third fluid in the at least one second microreactor;
initiating a reaction between an activated flavan derivative of the first product fluid and the flavan derivative of the third fluid acting as a nucleophile;
mixing the second product fluid and the fourth fluid in the at least one third microreactor;
stopping a reaction of the second product fluid by using the base; and
producing an oligomer containing the flavan derivatives bonded to each other.

11. The flavan oligomer production method according to claim 7, wherein the flavan derivative is a monomer including one flavan skeleton or an oligomer including two or more flavan skeletons.

12. The flavan oligomer production method according to claim 7, wherein the flavan derivative is a compound represented by the following general formula (1) or (2): 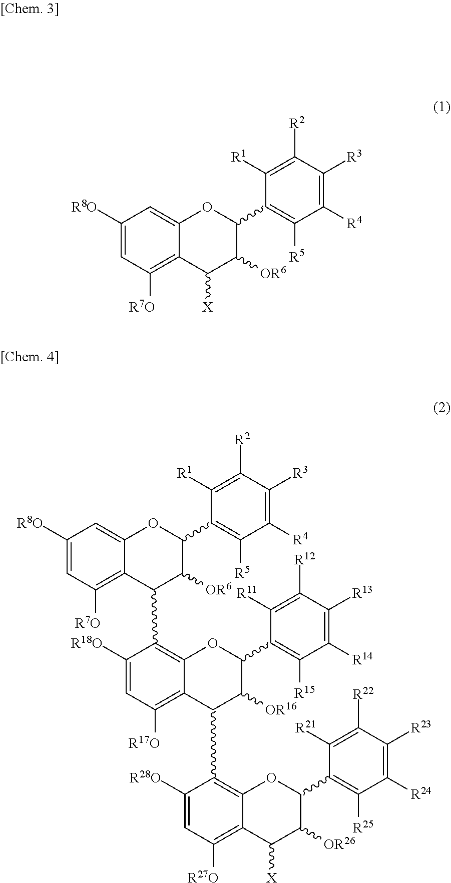
wherein in the general formula (1), R¹ to R⁵ each independently represent a hydrogen atom, a hydroxy group, an alkoxy group, or a substituent represented by OR⁹; R⁶ represents a hydrogen atom, a hydrocarbon group which may include a substituent, an alkoxyalkyl group, an acyl group, a silyl group, or a galloyl group; R⁷ to R⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration, and
wherein in the general formula (2), R¹ to R⁵, R¹¹ to R¹⁵, and R²¹ to R²⁵ each independently represent a hydrogen atom, a hydroxy group, an alkoxy group, or a substituent represented by OR⁹; R⁶, R¹⁶, and R²⁶ represents a hydrogen atom, a hydrocarbon group which may include a substituent, an alkoxyalkyl group, an acyl group, a silyl group, or a galloyl group; R⁷ to R⁸, R¹⁷ to R¹⁸, and R²⁷ to R²⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration; n represents an integer of 0 or larger than 0.

13. The flavan oligomer production system according to claim 2, wherein the flavan derivative is a monomer including one flavan skeleton or an oligomer including two or more flavan skeletons.

14. The flavan oligomer production system according to claim 3, wherein the flavan derivative is a monomer including one flavan skeleton or an oligomer including two or more flavan skeletons.

15. The flavan oligomer production system according to claim 4, wherein the flavan derivative is a monomer including one flavan skeleton or an oligomer including two or more flavan skeletons.

16. The flavan oligomer production system according to claim 2, wherein the flavan derivative is a compound represented by the following general formula (1) or (2): 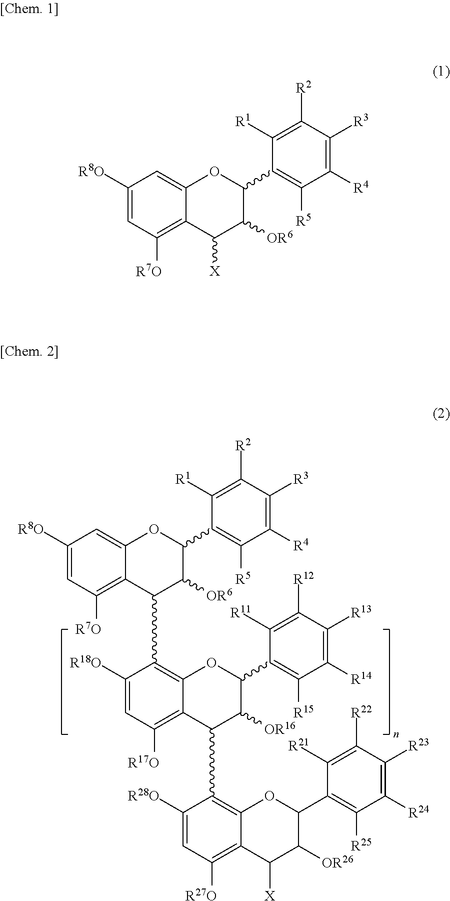
wherein in the general formula (1), R¹ to R⁵ each independently represent a hydrogen atom, a hydroxy group, an alkoxy group, or a substituent represented by OR⁹; R⁶ represents a hydrogen atom, a hydrocarbon group which may include a substituent, an alkoxyalkyl group, an acyl group, a silyl group, or a galloyl group; R⁷ to R⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration, and
wherein in the general formula (2), R¹ to R⁵, R¹¹ to R¹⁵, and R²¹ to R²⁵ each independently represent a hydrogen atom, a hydroxy group, an alkoxy group, or a substituent represented by OR⁹; R⁶, R¹⁶, and R²⁶ represents a hydrogen atom, a hydrocarbon group which may include a substituent, an alkoxyalkyl group, an acyl group, a silyl group, or a galloyl group; R⁷ to R⁸, R¹⁷ to R¹⁸, and R²⁷ to R²⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration; n represents an integer of 0 or larger than 0.

17. The flavan oligomer production system according to claim 3, wherein the flavan derivative is a compound represented by the following general formula (1) or (2): 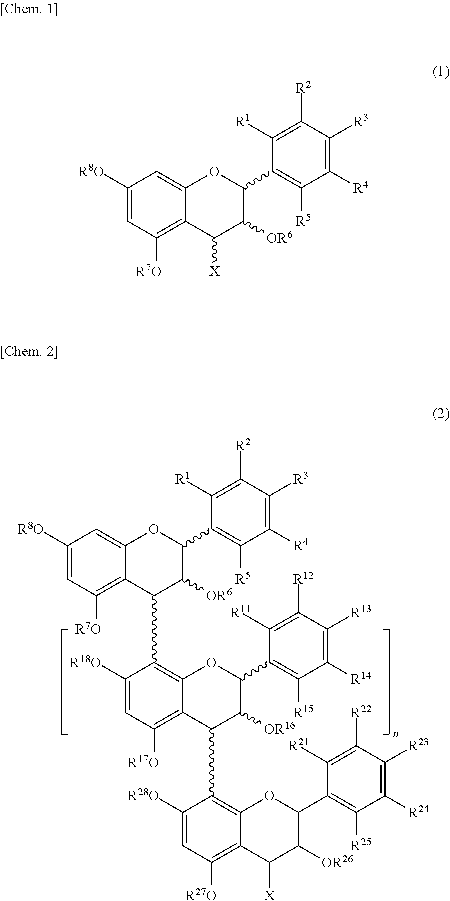
wherein in the general formula (1), R¹ to R⁵ each independently represent a hydrogen atom, a hydroxy group, an alkoxy group, or a substituent represented by OR⁹; R⁶ represents a hydrogen atom, a hydrocarbon group which may include a substituent, an alkoxyalkyl group, an acyl group, a silyl group, or a galloyl group; R⁷ to R⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration, and
wherein in the general formula (2), R¹ to R⁵, R¹¹ to R¹⁵, and R²¹ to R²⁵ each independently represent a hydrogen atom, a hydroxy group, an alkoxy group, or a substituent represented by OR⁹; R⁶, R¹⁶, and R²⁶ represents a hydrogen atom, a hydrocarbon group which may include a substituent, an alkoxyalkyl group, an acyl group, a silyl group, or a galloyl group; R⁷ to R⁸, R¹⁷ to R¹⁸, and R²⁷ to R²⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration; n represents an integer of 0 or larger than 0.

18. The flavan oligomer production system according to claim 4, wherein the flavan derivative is a compound represented by the following general formula (1) or (2): 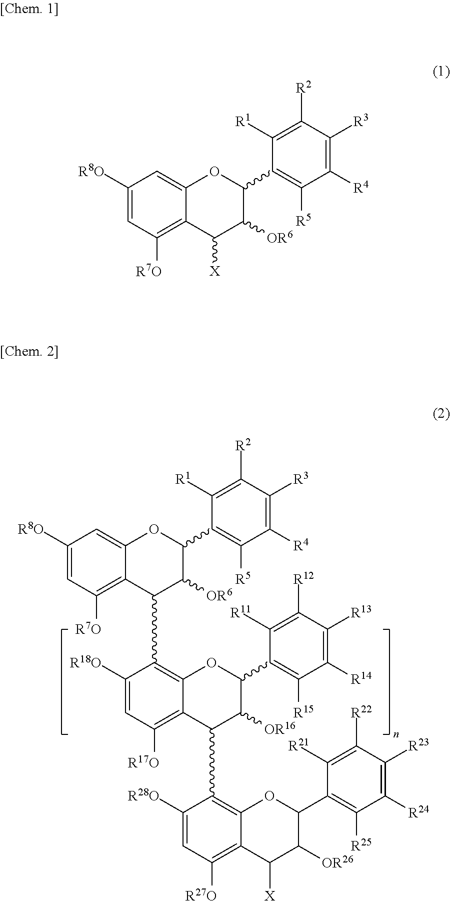
wherein in the general formula (1), R¹ to R⁵ each independently represent a hydrogen atom, a hydroxy group, an alkoxy group, or a substituent represented by OR⁹; R⁶ represents a hydrogen atom, a hydrocarbon group which may include a substituent, an alkoxyalkyl group, an acyl group, a silyl group, or a galloyl group; R⁷ to R⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration, and
wherein in the general formula (2), R¹ to R⁵, R¹¹ to R¹⁵, and R²¹ to R²⁵ each independently represent a hydrogen atom, a hydroxy group, an alkoxy group, or a substituent represented by OR⁹; R⁶, R¹⁶, and R²⁶ represents a hydrogen atom, a hydrocarbon group which may include a substituent, an alkoxyalkyl group, an acyl group, a silyl group, or a galloyl group; R⁷ to R⁸, R¹⁷ to R¹⁸, and R²⁷ to R²⁸ each independently represent a hydrogen atom or a substituent represented by R⁹; R⁹ represents a hydrocarbon group which may include a substituent, an alkoxyalkyl group, an acyl group, or a silyl group; X represents a hydrocarbon group which may include a substituent, a halogen atom, or a substituent containing at least one heteroatom selected from the group consisting of N, O, and S; the at least one heteroatom is bonded to a ring-forming atom of the C ring; and the wavy line is a single bond indicating an R-configuration or an S-configuration; n represents an integer of 0 or larger than 0.

19. The flavan oligomer production method according to claim 8, wherein the flavan derivative is a monomer including one flavan skeleton or an oligomer including two or more flavan skeletons.

20. The flavan oligomer production method according to claim 9, wherein the flavan derivative is a monomer including one flavan skeleton or an oligomer including two or more flavan skeletons.
